(19) United States
(12) Patent Application Publication
(10) Pub. No.: US 2025/0000369 A1
(43) Pub. Date: Jan. 2, 2025

(54) ARTIFICIAL INTELLIGENCE ENHANCED REAL-TIME PHYSIOLOGICAL PARAMETER MEASUREMENT AND MONITORING SYSTEM

(71) Applicant: AION BIOSYSTEMS, INC., Darien, CT (US)

(72) Inventors: Thomas Blackadar, Auburn, NH (US); Peter Costantino, Westport, CT (US); Kristen Warren, Waltham, MA (US); Sam Cheuvront, Franklin, MA (US); David Goodall, Chelmsford, MA (US); D. Alexander Colburn, Lowell, MA (US)

(21) Appl. No.: 18/679,940

(22) Filed: May 31, 2024

Related U.S. Application Data
(63) Continuation-in-part of application No. 18/678,366, filed on May 30, 2024.
(60) Provisional application No. 63/505,427, filed on May 31, 2023.

(51) Int. Cl. A61B 5/01; A61B 5/00; G01K 13/20; G01K 15/00; G16H 40/67; G16H 50/30

(52) U.S. Cl. CPC A61B 5/01; A61B 5/0008; A61B 5/0022; A61B 5/412; A61B 5/6833; A61B 5/7267; A61B 5/7275; A61B 5/746; G01K 13/20; G01K 15/005; G16H 40/67; G16H 50/30; A61B 2560/0252; A61B 2562/0271

(57) ABSTRACT

The present invention relates to a system and method for, for example, periodically collecting physiological parameters in real-time from a plurality of subjects, for example temperature from cancer patients, heartbeat rates from persons being treated for coronary conditions, physiological orientation information mechanical shock and related parameters, for example from football players in danger of head trauma, and other physiological measurements which it real-time convey useful information. This information is coupled to a system which integrates the information, subjects it to criteria (for example doctor-specified dangerous condition criteria), communicates information and, optionally provides alarms to clinicians. The reliability of the monitoring function is enhanced by an artificial intelligence algorithm in which one of the features is a predicted temperature based upon a predicted oral temperature which was developed using a model with a relationship defined by the comparison of real-world oral temperature measurements to skin temperature measurements and then using that prediction as a feature input into an artificial intelligence algorithm together with additional features comprising clinically collected health information in publicly available databases.

### CROSS REFERENCE TO RELATED APPLICATIONS

This application claims the benefit of Provisional Patent Application No. 63/505,427, filed May 31, 2023, and entitled Secure Systems for Measuring Physiological Parameters, the disclosure of which is hereby incorporated herein by reference. This application also incorporates by reference U.S. patent application Ser. No. 18/678,366 entitled Power Conserving Secure Systems for Vital Sign Monitoring filed on May 30, 2024.

### TECHNICAL FIELD

The invention relates to devices, systems and methods for measuring physiological parameters in the human body using a skin applied device, where outputs are subjected to a predictive algorithm and used it to drive and artificial intelligence classification tree.

### STATEMENT REGARDING FEDERALLY SPONSORED RESEARCH OR DEVELOPMENT

(Not applicable)

### BACKGROUND OF THE INVENTION

Physiological parameters indicating the functioning of the human body is of recognized importance. In some cases real-time monitoring is also recognized to be of value. For example, the taking of human body temperatures is a well-recognized and highly valuable medical tool with widespread uses ranging from first-aid, emergency detection, diagnosis, reducing disease propagation, and a wide range of patient monitoring functions. Accordingly, there are a wide range of suitable devices for taking human temperatures, for example, digital, ear, strip, mercury and infrared thermometers. Likewise, human body temperatures may be taken in a variety of locations on the human body. Both the temperature measurement method employed and the location where the reading is taken affects results.

Other instances of physiological parameters recognized as being important include information relating to the magnitude of physical shock to the body, heart rate, blood pressure, and so forth.

### SUMMARY OF THE INVENTION

The invention may be applied to numerous physiological parameters, as noted above. However, for illustrative purposes, the present specification focuses largely on body temperature. With that in mind, body core temperature is the gold standard by which any temperature measurement method may be judged. It is, by and large, the most accurate indicator of the state of body defensive actions and other factors correlated to the numerous factors of human body condition and/or disease. Nevertheless, in accordance with the invention it has been recognized by the inventors that body core temperature, for all its reliability in indicating the condition of the human body, is not the most useful temperature reading, for example in the clinical setting, because numerous factors, not related to the core temperature, affect the value of clinically taken temperature values, for example, oral thermometer readings.

In accordance with the invention, a method is provided for taking a reading of a physiological parameter (such as a patient's body temperature), which in daily practice suffers from unreliability-causing factors, by generating a raw reading (for example, from a temperature transducer coupled to and ideally in physical contact with the skin of a patient). The raw reading is processed in accordance with the invention to enable, in the case of the example, reliable body temperature reporting to medical professionals, as well as to allow for the detection of a dangerous condition and the sending of alarms. More particularly, in accordance with the preferred embodiment, the raw reading is used to generate a body core temperature prediction.

The present invention stems from the inventors' recognition that apart from providing objective/quantifiable data, the purpose of any physiological measurement is thwarted if it does not fully harness the experience knowledge set of medical professionals such as doctors, nurses and technicians, and the invention achieves this by channeling patient data, clinician practices and multiple variables into the system. In accordance with the invention it has also been recognized that the experience knowledge set of medical professionals is largely the product of oral temperature readings in the context of the clinical situation which embodies not only the measured, for example, oral, temperature of the patient, but also numerous other factors including ambient conditions, the particular disease or condition being treated, the position of the patient, and numerous other factors observed by or known to the medical professional. These factors have been recognized by the inventors, in accordance with the invention, to have an effect on the temperature measured by the medical professional, and more importantly all the baseline for clinician judgments. The result is that repeatable and accurate automatic parameter readings are less useful than parameter readings processed using statistical clinical information, which statistically reflect what happens in the real world when the clinician takes a measurement clinically.

While there are numerous instances where similar fact patterns occur, consider a patient suffering from nasal congestion who is forced to breathe through the mouth. This could result in somewhat lower temperature readings. Such circumstance might likely be apparent to, for example, a nurse who might, without even knowing it, unconsciously factoring that fact into an assessment of an oral temperature reading.

The most commonly used temperature measurement method relied upon by medical professionals is a reading of oral temperature taken using a digital thermometer. As alluded to above, measurements taken using such a device, particularly in the context of the medical setting, suffer from numerous inaccuracies and, accordingly, have only limited application. Nevertheless, in accordance with the invention it has been recognized that replication of that type of reading, with all its problems, can be useful.

Thus, in accordance with the invention, the raw reading from the skin contacting temperature transducer, in the case of the example of temperature reading, is used to generate a body core temperature prediction and an oral temperature prediction. Further in accordance with the invention, both predictions are utilized in a manner designed to maximize the possible level of care for the patient while at the same time providing a device will have a long useful life using an onboard battery.

More particularly, the same is achieved by processing of temperature transducer signals to generate multiple information signals which drive displays, machine learning, and alarm systems, by providing a diversity of patient evaluation and monitoring information, while at the same time utilizing a large data set and artificial intelligence algorithm, thus enabling medical professionals to take advantage of their individual experience knowledge set with a selection of information and stimuli.

At the same time, in accordance with the invention, clinician generated data may, optionally, be used, after associating the same with appropriate transducer generated data to check system reliability and improve the algorithm predicting the various temperatures generated and/or used by the system.

It is further noted that the system example is keyed, inter alia, toward generation of an oral temperature prediction. Oral temperature is the most commonly used clinical method as noted above. However, the algorithm may be adjusted to output another parameter instead of oral temperature, such as an infrared temple temperature, should changes in medical practice evolved. In accordance with one preferred embodiment of the invention, the clinician may optionally select one, or optionally more than one, type of temperature reading prediction, for example for display on a device accessible to the clinician, and/or on different devices.

To summarize, in the example, a sensor (for example, a sensor connected to the body for an extended period days or months long) detecting temperature, heart rate, blood pressure or another parameter, produces a reading. That reading is converted, in the example, to an oral temperature reading using a conversion equation which is the product of a best fit approximation of a pair of clinically collected data point pairs. Each data point pair comprises a sensor collected data point (in the example skin temperature data point) and a substantially simultaneous clinically collected data point (in the example an oral temperature taken with a digital thermometer). These data point pairs (with outliers and unreliable data removed) may be plotted on an x-y axis and a function (such as a line, sigmoid or the like) fitted to the points to generate the conversion equation.

This statistically-converted sensor reading may, in accordance with the invention, then be processed with a clinically collected database, for example a public database such as that collected by the NIH, relating, at least in part, to a particular disease, such as sepsis, using artificial intelligence techniques with selected parameters in the public database constituting input features for an artificial intelligence algorithm, and the statistically-converted sensor readings coming from a particular patient, and other features associated with that particular patient, all being used as features to protect the likelihood of the onset of the condition (in this example, sepsis).

More particularly, a set of diagnostic features other than temperature and a physiological temperature are provided as features, the physiological temperature being a predicted temperature based on a function defining the relationship between oral temperatures taken by clinicians (for example with a simple digital thermometer typically used in hospitals and medical facilities) and a skin temperature-based electronically collected reading. The function is used to convert real-time physiological skin temperature measurements to predicted oral temperatures, for the purpose of feeding that data to an artificial intelligence algorithm as one feature together with the patient's other physiological parameter(s) as other features. The artificial intelligence algorithm is generated by being trained on clinical data from a plurality, for example, large number, of patients.

The use of the inventive approach to measure a parameter, for example core temperature, and alarms and actions in response thereto, separate from a user-friendly interface exhibiting oral temperature and which dovetails with clinical experience which may take into account experiential variables that are hard to quantify and may not even be consciously recognized by the clinician, including intuition, experience, reflexive reaction, and others, such as clinician observations, patient position, etc. Viewed another way, oral temperatures, for example, may vary from core temperature because of numerous factors which are subconsciously taken into account by clinicians in their assessment of temperature. Thus, the oral predictions derived from core temperature and potentially historical values will give a reading which matches clinician experience. Putting in additional variables like disease or sex will take into account factors affecting the transition, for example the tendency of a patient suffering from a respiratory infection to bring to the mouth, thus lowering temperatures, or the tendency of the patient suffering from a disease which causes a low level of energy and thereby to cause the patient to be laying down, altering the relationship between core temperature and oral temperature.

The inventive method and apparatus generally comprises an apparatus that measures temperature on a mammal such as a human in accordance with the present invention. The apparatus comprises a temperature measurement device with an onboard device power source to powers its components. The apparatus further comprises a clock having a first high speed output, and a second low speed output. In preferred embodiments, the high speed output is at least 10 times as fast as the low speed output. The apparatus further comprises a data memory sector and a digital processor, where the digital processor is responsive to the high-speed output to generate a storage trigger signal. The apparatus further comprises a temperature measurement transducer responsive to the storage trigger signal from the digital processor, to periodically collect a temperature measurement and to couple the collected temperature measurement to the data memory sector to store the collected temperature measurement in the data memory sector to accumulate data in the data memory sector as an accumulation of data in the form of a plurality of data points. The apparatus further comprises wireless transceiver having an input and an output digital processor that responds to the low-speed output by generating a transmission trigger signal. This signal is coupled to the data memory sector. The transmission trigger signal then prompts the data memory sector to transfer the accumulated data to the input of the wireless transceiver for transmission. The apparatus further comprises a non-volatile memory sector that contains a program of onboard instructions. These instructions control the digital processor, directing the temperature measurement transducer to store data points in the data memory sector and to cause the wireless transceiver to transmit these data points. The apparatus further comprises a chassis member that supports the power source, clock, data memory sector, digital processor, temperature measurement transducer and wireless transceiver. The apparatus further comprises means to access a publicly accessible network. The apparatus further comprises a wireless repeater that receives the accumulation of data output from the wireless transceiver and couples the data to the publicly accessible network.

The apparatus further comprises a server equipped with a central processing unit (CPU) that connects to the publicly accessible network to receive and accumulate data points Additionally, the server features a temperature data memory to store these data points. The server is further equipped with a non-volatile memory containing server instructions, directing the CPU to receive, store, and transmit the data points to at least one user. The apparatus further comprises a non-volatile temperature conversion program memory with a program of temperature conversion instructions for converting the data points to predicted physiological temperature readings for the transmission to at least one user.

In a preferred embodiment of the invention, the non-volatile temperature conversion program memory does not have to be located on the chassis or powered by the onboard device power source.

In another preferred embodiment of the invention, the inventive apparatus further comprises an adhesive member to secure the chassis member to the chest of a patient to predict oral temperature.

In another preferred embodiment of the invention, the inventive apparatus can be used to predict oral temperature by using a mobile personal computing device coupled to the publicly accessible network as a transceiver while the non-volatile temperature conversion program is coupled to and run on the personal computing device.

In another preferred embodiment of the invention, a smartphone could function as wireless repeater where the non-volatile memory containing the temperature conversion program could be connected to and executed by a digital signal processor within the smartphone.

In another preferred embodiment of the invention, the inventive apparatus would a predict an oral temperature and have the chassis member comprise a waterproof housing.

The inventive apparatus utilizes the program of temperature conversion instructions to predict oral temperature by instructing calculation to convert data points into an oral temperature reading.

In another preferred embodiment, the inventive apparatus predicts oral temperature using a program of temperature conversion instructions. These instructions convert data points into predicted oral temperature readings by collecting a variety of clinical calibration skin temperature measurements from skin-mounted temperature measurement transducers, along with clinical calibration oral temperature measurements. These combined measurements, comprising a plurality of data points, are then used to fit a function to the plurality of data points.

An alternative embodiment of invention comprises an apparatus that measures temperature comprises a temperature measurement device which comprises a device power source that powers components of the temperature measurement device. The power source may comprise a dependent battery or a capacitor. The apparatus further comprises a clock having a first high speed output, and a second low speed output. In preferred embodiments, the high speed output is at least 10 times as fast as the low speed output. The apparatus further comprises a data memory sector and a digital processor, where the digital processor is responsive to the high-speed output to generate a storage trigger signal. The apparatus further comprises a temperature measurement transducer responsive to the storage trigger signal from the digital processor, to periodically collect a temperature measurement and to couple the collected temperature measurement to the data memory sector to store the collected temperature measurement in the data memory sector to accumulate data in the data memory sector as an accumulation of data in the form of a plurality of data points. The apparatus further comprises wireless transceiver having an input and an output digital processor that responds to the low-speed output by generating a transmission trigger signal. This signal is coupled to the data memory sector. The transmission trigger signal then prompts the data memory sector to transfer the accumulated data to the input of the wireless transceiver for transmission. The apparatus further comprises a non-volatile memory sector that contains a program of onboard instructions. These instructions control the digital processor, directing the temperature measurement transducer to store data points in the data memory sector and to cause the wireless transceiver to transmit these data points. The apparatus further comprises a chassis member that supports the power source, clock, data memory sector, digital processor, temperature measurement transducer and wireless transceiver. The apparatus further comprises means to access a publicly accessible network. The apparatus further comprises a wireless repeater that receives the accumulation of data output from the wireless transceiver and couples the data to the publicly accessible network. The apparatus further comprises a server equipped with a central processing unit (CPU) that connects to the publicly accessible network to receive and accumulate data points

Additionally, the server features a temperature data memory to store these data points. The server is further equipped with a non-volatile memory containing server instructions, directing the CPU to receive, store, and transmit the data points to at least one user. The apparatus further comprises a non-volatile temperature conversion program memory with a program of temperature conversion instructions for converting the data points to predicted physiological temperature readings for the transmission to at least one user. The apparatus further comprises a non-volatile temperature conversion program memory with a program of temperature conversion instructions for converting the data points to predicted physiological temperature readings for the transmission to at least one user. The program of temperature conversion instructions, convert data points into predicted physiological temperature readings and is generated through the collection of two sets of measurements: clinical calibration skin temperature measurements obtained from skin-mounted temperature measurement transducers, and clinical calibration physiological temperature measurements. These measurements, comprising a plurality of data points, are then used to fit a function to the plurality of data points.

In preferred embodiments, the inventive apparatus' device power source is a battery.

An inventive method for measuring and monitoring temperature is provided using a temperature measurement device. The method includes using a device power source to power components of the temperature measurement device; operating a clock to produce a first high speed output and a second low speed output, where the high speed output of the clock being at least 10 times as fast as the low speed clock output.; reading and writing to a data memory sector; generate a storage trigger signal using a digital processor responsive to the high-speed output of the clock; periodically collecting a temperature measurement using a temperature measurement transducer responsive to the storage trigger signal from the digital processor; coupling couples the collected temperature measurement to the data memory sector to store the collected temperature measurement in the data memory sector to accumulate data in the data memory sector as an accumulation of data in the form of a plurality of data points; generating in response to the low speed output, a transmission trigger signal using the digital processor, coupling the transmission trigger signal to the data memory sector, causing the data memory sector to couple for transmission where the transmission of the couple may consist of the accumulation of data stored in the data memory sector to the input of a wireless transceiver having an input and an output; using a non-volatile memory sector with a program of onboard instructions for controlling the digital processor, causing the temperature measurement transducer to store the data points in the data memory sector to cause a wireless transceiver to transmit the data points; using a chassis member to support the power source, the clock, the data memory sector, the digital processor, the temperature measurement transducer and the wireless transceiver' coupling using a wireless repeater to couple to a publicly accessible network for example, the accumulation of data output from the wireless transceiver; receiving the accumulation of data using a server coupled to the publicly accessible network by using a server central processing unit to receive and store the data points using a temperature data memory coupled to the central processing unit of the server and may receive, storing and transmitting to at least one user the data points using a non-volatile server program memory with a program of server instructions; predicting an oral temperature reading for the transmission to at least one user using a non-volatile temperature conversion program memory with a program of temperature conversion instructions for converting the data points. The non-volatile temperature conversion program memory may or may not be located on the chassis or powered by the device power source.

In some embodiments, the non-volatile temperature conversion program memory is coupled to the server central processing unit and run on the server central processing unit.

In another preferred embodiment of the inventive method, an adhesive member is used to secure the chassis member to the chest of a patient.

In another preferred embodiment of the inventive method, a mobile personal computing device is used as a transceiver and is coupled to the publicly accessible network. The non-volatile temperature conversion program memory is coupled to personal computing device and runs on the personal computing device.

In another preferred embodiment of the inventive method, a smartphone is used as a wireless repeater. The non-volatile temperature conversion program memory is coupled to and run on a digital signal processor on the smartphone.

In another preferred embodiment of the inventive method, waterproofing is provided by the chassis member.

In another preferred embodiment of the inventive method, the program of temperature conversion instructions for converting the data points to predicted oral temperature readings may instruct a calculation.

In accordance with the present invention, the method comprises taking a temperature measurement using a temperature measurement device; the temperature measurement device uses a device power source that powers components of the temperature measurement device; the temperature measurement device operates a clock to produce a first high-speed output and a second low-speed clock output, the high-speed output being, for example, at least 10 times as fast as the low-speed clock output; the temperature measurement device reads and writes to a data memory sector; the temperature measurement device generates a storage trigger signal using a digital processor that is responsive to the high-speed clock output; the temperature measurement device periodically collects a temperature measurement using a temperature measurement transducer that is responsive to the storage trigger signal from the digital processor; the temperature measurement device couples the collected temperature measurement to the data memory sector to store the collected temperature measurement in the data memory sector, accumulating data in the form of a plurality of data points; the temperature measurement device generates, in response to the low-speed clock output, a transmission trigger signal using the digital processor; the temperature measurement device couples the transmission trigger signal to the data memory sector; the transmission trigger signal causes the data memory sector to transmit the accumulated data stored in the data memory sector to the input of a wireless transceiver having an input and an output; the temperature measurement device uses a non-volatile memory sector with a program of onboard instructions for controlling the digital processor; the digital processor causes the temperature measurement transducer to store the data points in the data memory sector, which then causes a wireless transceiver to transmit the data points; the temperature measurement device uses a chassis member to support the power source, clock, data memory sector, digital processor, temperature measurement transducer, and the wireless transceiver; the method couples the accumulation of data output from the wireless transceiver to a publicly accessible network using a wireless repeater; the method receives the accumulation of data using a server coupled to the publicly accessible network by using a server central processing unit to receive and store the data points; the method uses a temperature data memory coupled to the central processing unit of the server; the method receives, stores, and transmits the data points to at least one user using a non-volatile server program memory with a program of server instructions; the method predicts an oral temperature reading for transmission to at least one user using a non-volatile temperature conversion program memory with a program of temperature conversion instructions for converting the data points and accumulated data stored in the data memory sector to the input of a wireless transceiver having an input and an output; the temperature measurement device uses a non-volatile memory sector with a program of onboard instructions for controlling the digital processor. The digital processor causes the temperature measurement transducer to store the data points in the data memory sector, which then causes a wireless transceiver to transmit the data points; the temperature measurement device uses a chassis member to support the power source, clock, data memory sector, digital processor, temperature measurement transducer, and the wireless transceiver.

In another preferred embodiment of the invention, the program of temperature conversion instructions for converting the data points to predicted oral temperature readings is generated by collecting a plurality of collected clinical calibration skin temperature measurements from skin mounted temperature measurement transducers. The program may collect a plurality of collected clinical calibration oral temperature measurements, the calibration skin temperature measurements and the clinical calibration oral temperature measurements. The temperature measurements comprise a plurality of data points, and fitting a function to the plurality of data points.

In another preferred embodiment of the invention, the physiological measurement is core temperature. The temperature conversion instructions are a function of clinical skin temperature measurements and clinical core temperature measurements. The temperature instructions may comprise oral temperature conversion instructions for converting core temperature measurement readings from the temperature measurement transducer to oral temperature measurement readings. The oral temperature conversion instructions is based upon clinical skin temperature measurements and clinical oral temperature measurements.

The inventive apparatus may comprise a high temperature alarm circuit for signaling a fever condition. The high temperature alarm circuit is triggered by core temperature measurements exceeding a fever indicating threshold.

In another preferred embodiment of the invention, the inventive apparatus may comprise a high temperature alarm circuit for signaling a fever condition. The high temperature alarm circuit is triggered by core temperature or converted oral temperature measurements exceeding a fever indicating threshold at least twice in sequential readings.

In another preferred embodiment of the invention, the non-volatile temperature conversion program memory is coupled to and run on the server central processing unit.

In another preferred embodiment of the invention, the chassis comprises a flexible frame having a bottom and a top, with the flexible frame including a peripheral portion that extends at least partially around and is positioned at the periphery of the frame. Additionally, the flexible frame features an inner portion that at least partially surrounds the circuit board, while also being surrounded by the peripheral portion. Further, the flexible frame includes an intermediate portion positioned between the peripheral portion and the inner portion, with a skin-facing bottom wall secured at the bottom of the frame, and an ambient-facing top wall secured at the top of the frame. Within this flexible frame, an onboard device power source, clock, data memory sector, digital processor, temperature measurement transducer, wireless transceiver, and non-volatile memory sector are positioned between the top wall and bottom wall.

In another preferred embodiment of the invention, the intermediate portion has a thinner portion thinner than the peripheral portion. The thinner portion is flexible enough to allow movement of the intermediate portion between the peripheral portion and the inner portion. The frame includes a thinned out area within the circumference of the device promoting adhesion and sealing. Inwardly and outwardly facing temperature sensors measure, respectively, skin temperature and ambient temperature, with ambient temperatures providing information relating to the reliability of the skin temperature measurement.

In another preferred embodiment of the invention, a sealed compartment is formed by the peripheral portion and the bottom and top walls. The onboard device power source, clock, data memory sector, digital processor, temperature measurement transducer, wireless transceiver, and the non-volatile memory sector is positioned within the compartment.

In another preferred embodiment of the invention, the clock comprises two separate clocks.

A method of measuring a human physiological temperature in accordance with the present invention comprises using the inventive apparatus and mounting it below the human clavicle.

In another preferred embodiment of the invention, the onboard device power source comprises a power source that is selected from the group consisting of a battery or a capacitor.

The inventive apparatus serves as a diagnostic tool designed to generate an early prediction of the likely onset of sepsis in a patient. This diagnostic tool comprises a classifier and a set of diagnostic features, excluding the patient's temperature and oral temperature. Stored in a non-transitory computer-readable medium, these instructions, when executed by the server's central processing unit, prompt the unit to provide the set of diagnostic features, omitting the patient's physiological temperature, as an input to the classifier. Simultaneously, it includes the patient's physiological temperature as an additional input feature. The classifier is trained with data from a multitude of individuals who have experienced sepsis.

Utilizing both the set of diagnostic features and the physiological temperature feature as inputs, the tool assesses the likelihood of sepsis development. It then generates an output indicating whether there are indications that the subject is likely to develop sepsis.

In another preferred embodiment of the invention, the diagnostic features other than oral temperature comprise one or more features selected from the group consisting of heart rate, gender, age, and ethnicity.

In another preferred embodiment of the invention, the physiological temperature feature is a predicted physiological oral temperature.

In another preferred embodiment of the invention, the program for converting data points into predicted oral temperature readings is developed by gathering numerous clinical calibration skin temperature measurements from skin-mounted temperature transducers. This collection, which includes both clinical calibration oral temperature measurements and skin temperature measurements, yields a plethora of data points. Subsequently, a function is fitted to these data points.

The inventive method assesses the likelihood of an individual developing sepsis by utilizing a classifier and a set of diagnostic features excluding temperature and oral temperature. Stored in a non-transitory computer-readable medium, these instructions prompt the server's central processing unit to provide the set of diagnostic features, excluding physiological temperature, as input to the classifier. Additionally, the physiological temperature of the patient is provided as a separate input feature. Trained with data from numerous individuals who have experienced sepsis, the classifier evaluates the likelihood of sepsis by incorporating both the set of diagnostic features and the physiological temperature. Subsequently, it generates an output indicating whether there are signs that the subject may develop sepsis.

In another preferred embodiment of the invention, the temperature measurement transducer faces in one direction toward the general direction of the skin of the user and is coupled to collect a skin temperature reading. The temperature measurement transducer comprises an additional temperature transducer facing generally away from the skin of the user toward the ambient and is positioned to collect an ambient temperature reading. When the ambient temperature reading is below a low threshold value or above a high threshold value, the collected skin temperature reading is discarded or devalued.

In another preferred embodiment, the inventive apparatus consists of a diagnostic tool for generating an early prediction of the likely onset of sepsis in a patient. This tool includes a classifier coupled to receive and operate on a set of diagnostic features other than the temperature associated with the patient. A derived temperature reading comprises a predicted oral temperature derived from a patient's physiological temperature as an additional feature. The apparatus comprises a central processing unit, a temperature measurement device producing a physiological temperature output, and a non-transitory computer readable medium that stores instructions. When the instructions are executed by the central processing unit, they cause the central processing unit to convert the physiological temperature output of the temperature measurement device to a predicted oral temperature reading. The execution of the set of instructions implementing a function between physiological temperature output and predicted oral temperature readings causes the central processing unit to generate the predicted oral temperature reading. This function is derived by collecting data comprising a plurality of sets of clinical oral temperature readings and associated clinical temperature readings more reliable than clinical oral temperature readings, and fitting the data to derive the function. Additionally, the instructions cause the central processing unit to provide the set of diagnostic features other than temperature associated with the patient and the predicted oral temperature as an input to the classifier. The classifier is trained with data corresponding to data in the set from a plurality of individuals who have had sepsis. The instructions further cause the central processing unit to evaluate, with the classifier, the likelihood of sepsis by using both a set of diagnostic features other than temperature and the derived oral temperature as features. Finally, the instructions cause the central processing unit to generate an output indicating whether the patient is likely to develop sepsis.

### BRIEF DESCRIPTION OF THE DRAWINGS

The operation of the inventive temperature measurement and monitoring system will become apparent from the following description taken in conjunction with the drawings, in which:

FIG. 1 is a hardware block diagram illustrating a clinician fixed facility portal, and individual mobile portals for patients and clinicians in a general publicly accessible network based implementation of the present invention;

FIG. 2 is a hardware block diagram illustrating the central server communication, control and artificial intelligence system infrastructure with the fixed and mobile portals of FIG. 1 in a general implementation of the present invention;

FIG. 3 is a hardware block diagram illustrating a fixed multi-patient treatment facility communicating with the central server of FIG. 2 in an optional and further elaboration of the system of the present invention, and, together with FIGS. 1 and 2, illustrates an implementation of a monitoring, communications and control system constructed in accordance with the present invention;

FIG. 4 is a flowchart illustrating the method of collecting and transmitting temperature measurements in accordance with the invention in the system illustrated in FIGS. 1-3;

FIG. 5 illustrates the inventive temperature measurement device in exploded perspective;

FIG. 6 illustrates the inventive temperature measurement device in perspective from the top of the device without the battery in place;

FIG. 7 illustrates the inventive temperature measurement device in perspective from the top of the device with the battery in place;

FIG. 8 is a bottom view of the inventive temperature measurement device in perspective;

FIG. 9 illustrates the inventive temperature measurement device of FIG. 5 in schematic cross-section;

FIG. 10 is a perspective drawing showing the inventive temperature measurement device mounted on the chest of a patient whose temperature is being monitored;

FIG. 11 is a hardware diagram of system to be used in combination with the hardware system of FIGS. 1-3 for determining the likely onset of sepsis in accordance with the invention;

FIG. 12 is a flowchart of methodology to be implemented in connection with the subsystem of FIG. 11;

FIG. 13 illustrates a mastoid-coupled shock detecting device in place on the head of the user, underneath the middle of the rear portion of the ear; and

FIGS. 14-15 together illustrate a multiple sensor peer to peer embodiment of the system of the present invention.

### DETAILED DESCRIPTION OF THE PREFERRED EMBODIMENTS

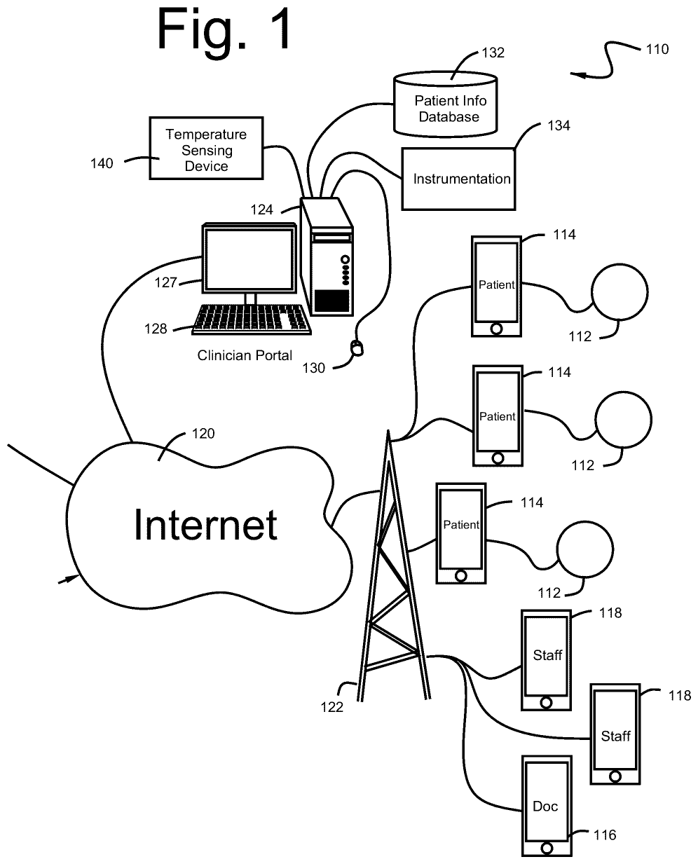

Referring to FIGS. 1-3, for purposes of ease of illustration focusing in on the example of temperature measurement, the inventive system 110 for the application of collecting body temperature information may be understood. System 110 comprises a plurality of temperature measurement devices 112 each associated with an individual patient. During operation of the system, temperature measurement devices 112 are each coupled to the smartphone 114 of an individual patient. Optionally, a single patient may be associated with multiple temperature measurement devices.

In similar fashion, doctors may access the system via smartphones 116, and supporting professionals such as nurses and paramedics (for example paramedics located on an ambulance rushing toward a patient wearing a temperature measurement device 112) may access the system through their respective smartphones 118. Smartphones 116 and 118, in turn, provide connectivity to the Internet 120, for example through a cellular telephone system tower 122.

Temperature measurement devices 112 are registered on the system at a clinician portal 124 which may be implemented on a personal computer, or for example by accessing the portal through a web browser. In accordance with the preferred embodiment, during use, temperature measurement devices are secured to the infraclavicular portion of the chest of the patient, roughly between the centerline of the chest (e.g. the sternum) and the left or right side of the chest in the horizontal direction. In the vertical direction temperature measurement device 112 is positioned a few inches below the clavicle and a few inches higher than the nipple. Temperature measurement device 112 may be secured by a medical grade adhesive tape, or specialized, for example, circular, medical grade adhesive tape members having a hole allowing an optional outward facing temperature sensor outside access with one less layer of insulation.

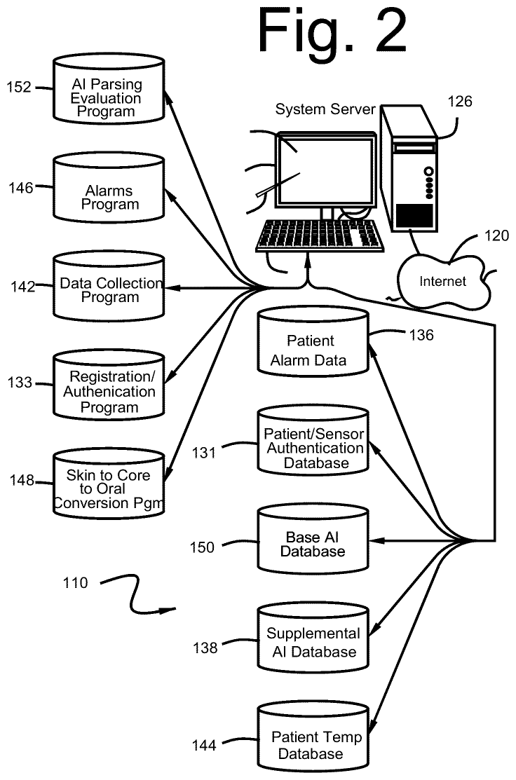

Clinician portal 124 is coupled, through the Internet 120, to the central server 126 of system 110 (FIG. 2). Central server 126 may be any suitable device, such as, for example, a high-speed personal computer with substantial onboard memory and computing power, or in the case of larger systems may comprise cloud based servers such as Google, Azure, and Amazon.

Clinician portal 124 may be a personal computer of the type employed and already existing in the clinician's facility, which may be a small office with a single doctor or a larger office housing multiple practices which may share the same clinician portal 124. Clinician portal 124 includes a hard drive on which is stored a browser application enabling the clinician to employ the system of the present invention as illustrated in FIGS. 1-3. Alternatively, the application may reside in the cloud on the server which operates and controls the inventive system for a number of users, who access the system through an Internet web browser.

Thus, computer 124 also includes a display 127 for the purpose of providing one or a number of input screens into which data may be entered through keyboard 128. Likewise, a mouse 130 may be used to enter data, navigate the program which enables use in the customary manner of the inventive system by the clinician.

Alternatively, the application enabling the clinician to employ the system of the present invention as illustrated in FIGS. 1-3 may be stored on central server 126.

Clinician portal 124 is used to register a patient on the inventive system. This is done by registering the patient on the clinician portal and then registering the temperature measurement device 112 to the patients account. Temperature measurement device 112 is paired to a patient smartphone 114. Once paired, temperature measurement device 112 data is sent to the central server computer 126. Such information is stored in database 131, for later use in authenticating data during the data collection patient monitoring process described below, and to continue the association of data from a replacement temperature measurement device associated with the patient when the prior device has been used for the planned period of time. The initial registration of the patient on system 110, and the registration of the device on system 110 both can occur during the same appointment. Alternatively, if, for example, the patient and or temperature measurement device 112 are being registered on the hospital or other facility system where the patient is expected to remain for an extended period of time, while a smartphone may be used, another device, optionally a Bluetooth-WIFI gateway device associated the facility may also be used.

If, for example, the patient and or temperature measurement device 112 are being registered on the system at a hospital, or other facility where the patient is expected to remain for an extended period of time, while a smartphone may be used, another device, optionally a Bluetooth device, may also be used. Such device may be associated, for example, with a hospital room.

In accordance with the invention, during registration, a particular temperature measurement device 112 is associated with a particular patient. This association is originated at the clinician portal 124 and saved at the central server in the cloud. When the system software located on server computer 124 receives communication from the mobile phone or gateway device the temperature measurement device 112, the unique identifier associated with the transducer on temperature measurement device 112 is checked against a database of authorized devices accessed by central server 126 (or optionally with other devices accessing the system). This ensures a proper identification of the sensor on the system. The system then pairs the device identification number to the patient and checks this number against the database of transducer identifier numbers and device identifier numbers.

If all the identifications are in order, only then does the system initialize the temperature measurement device 112 of the patient and capture its first sensor reading, for example in the case of a mobile device of the patient being coupled to temperature measurement device 112. The reading is captured from the advertising beacon of the temperature measurement device 112 associated with the patient. The advertising beacon has a unique ID of the sensor and the data payload necessary for upstream conversion to core temperature by the system server.

In accordance with the invention, registration accomplishes, as noted above, the extraction of a unique number associated with each measurement device 112, and this is used as a control and patient identifier until the device is replaced by a new device and the old device discarded by the patient. At this time the data collected by the old device remains associated with the patient and data collected by the new device with its new unique number is also associated with the patient's record going forward.

Typically, during registration, temperature measurement device 112 is registered to the clinician portal 124 to the patients account, and then clinician portal 124 passes to the central server the unique id of the patient as available on the system central server 126 by connecting to clinician portal computer 124 over Internet 120, pursuant to instructions in program 133.

During the same appointment, data relating to the patient is downloaded from a patient information database 132. This information is downloaded to central server 126 over the Internet 120. Also during that appointment, instrumentation 134, which is present in the facility of the clinician, may optionally be used to input information to central server 126. Information downloaded to central server 126 is maintained by the inventive system 110 in database 136, which contains such information as the patient's name, date of birth, medical history, a unique identifier associated with a temperature transducer on board temperature measurement device 112, as well as information associated with the patient and input by the clinician, such as high and low oral temperature values for alarm purposes, and/or, optionally, high and low body core temperatures to be associated with alarms to be sent to clinician team members.

Optionally, orally acquired patient temperature data taken by the practitioner may be input into the system 110 through clinician portal computer 124 and via Internet 120, augmenting database 138 associated with central server 126, with data collected by an oral thermometer 140 at the physician's office.

Alternatively, a patient may be registered on the system at his home or at another facility using a smartphone, such as a doctor's smart phone 116 connected to the clinician portal by Internet 120.

Referring to FIG. 2, central server 126 is connected to the clinician portal 124 (i.e. the computer of the clinician which has opened the browser and accessed the system over the Internet) through Internet 122 to receive input data from the clinician portal, including the setting of alarms and other data as noted above. Periodically, a program stored in memory 142 at central server 126 periodically, through Internet 120, receives the output of patient temperature measurement devices 112 to make a record of the patient's temperature. Such information is stored in database 136, from which it can be periodically read out for use by, for example, doctors, nurses and other clinicians.

In accordance with the invention, it is anticipated that developers will access the inventive system, for example in connection with the operation of a hospital, insurance company monitoring, and the like. Accordingly, limitations are imposed on the activities of such developed applications, for example the number of times temperature information on temperature measurement devices 112 may be accessed, i.e. when data scavenging occurs.

As will be described in detail below, the system 110 takes the information received when the system interrogates patient temperature measurement devices 112, and assesses the same against objective numerical criteria stored in patient alarm temperature setting database 136. Doctor-specified, patient specific alarm database 136 includes, for example, doctor-specified temperature boundaries which are compared to the periodic reading stored in database 144 under the control of a large program 146. For example, the doctor may specify that an alarm is to be sounded and the temperature reported when the patient's temperature measurement device 112 senses a temperature in excess of an oral temperature of 101.5° F. When such data is received, clinician portal 124 implements an alarm and sends the same over Internet 122, for example, the smartphone 118 of a nurse. Alternatively, the clinician portal computer may implement such alarm and send it to the smartphone of the patient.

To the extent that programming is to be customized to a particular patient, the same is desirably located on patient smart phone 114. The same is located on a smartphone, because a smart phone has a large amount of power available to it and placing the computing burden on the smart phone improves battery life in the inventive measurement device, thus not reducing the life of temperature measurement device 112 which may be powered, for example, by a small watch battery.

Other alarms can be set relating to temperatures maintained over a period of time, instructions may be implemented to override alarms unless certain conditions are met (for example, temperature being in an alarm range for less than three, for example, five-minute monitoring periods, or the like), or based upon other criteria. Likewise, doctor-specified patient alarm database 136 may have instructions with respect to sending alarm information to one or more specifically identified or role-identified persons, depending upon clinician instructions.

In accordance with the invention, the harnessing of the experience of clinicians is provided by presenting patient temperature information to the clinicians as an oral temperature reading of the type which would be obtained from a conventional mercury or electrical thermocouple thermometer (or, in principle, any desired standard temperature measurement device or method which has been used by the clinician). The oral temperature has the advantage of being a familiar number in the experience of the patient or clinician and the presentation of an oral measurement allows the clinician to rely upon experience consciously and subconsciously. Such conversion is provided by a skin temperature to core temperature to oral temperature conversion program stored in memory 148 associated with central server 126 when data is provided by Wifi Bluetooth gateway and Internet router. Alternatively, the core temperature to oral temperature conversion program may be associated with the clinician portal computer.

In practice, in accordance with the invention, skin temperature may be converted to core temperature to an acceptable degree of accuracy using the above conversion equation generated by curve fitting to non-discarded data point pairs corresponding to core temperature and a conventional oral temperature measurement.

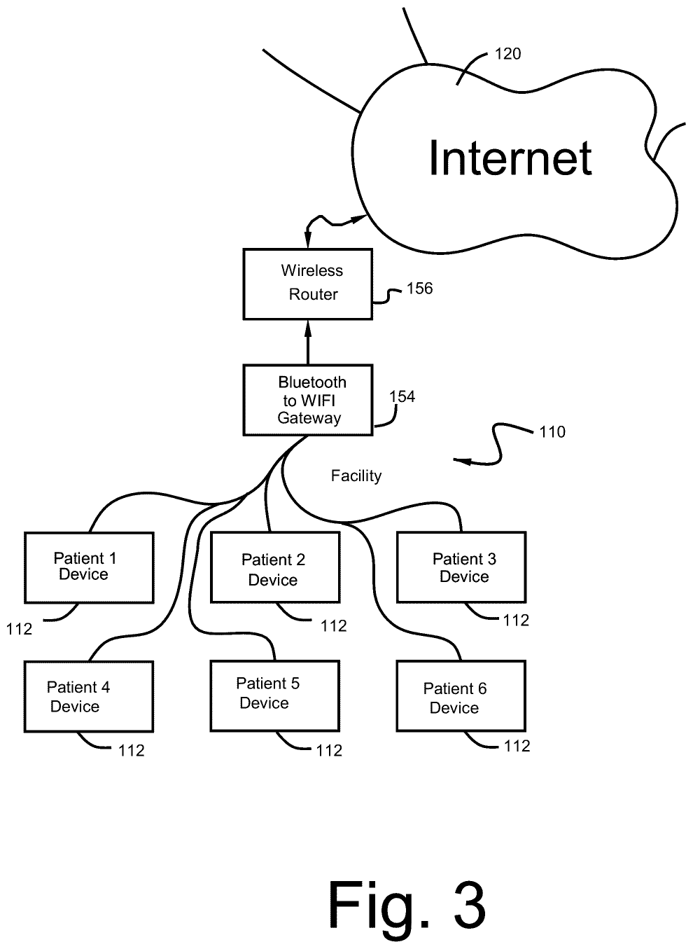

As illustrated in FIG. 3, patient devices 112 may be located in a treatment facility, such as a hospital where they are connected, for example, by Bluetooth or Wi-Fi to a repeater, for the coupling of information via the Internet 122 and from central server 126.

As an alternative to keeping all programming and calculation at the server, in principle, programming and calculation can be located at other locations in the system, for example on patient smart phones 114, staff smart phones 118, for example on the clinician's computer 124, and so forth. However, it is advantageous and in accordance with a preferred embodiment that the amount of software located on temperature measurement devices 112 be minimized, for reasons of power efficiency. Moreover, at the same time, for power conservation purposes, in accordance with the present invention, interrogation of temperature measurement devices 112 is only allowed limited number of times per hour. Alternatively, interrogation may be prohibited by the central server 126 and the transmission of all information from temperature measurement devices 112 initiated only by the temperature measurement device 112 itself. In accordance with a particularly preferred embodiment of the invention, it is contemplated that all software will be resident on computer 126. Accordingly, when the clinician desires to access the system, he or she opens the browser on computer 124 and accesses all functionality on computer 126.

In accordance with the present invention, readings are output by temperature measurement device 112 every five minutes (though they may be collected and stored on board device 112 more frequently, for example every 15 seconds depending upon the parameter being measured, with readings initiated by the temperature measurement device 112. This is the raw temperature reading in degrees centigrade output from the patient's temperature measurement device 112.

As noted above, the prediction of an oral temperature reading based upon a skin temperature reading, like other physiological measurements including heart rate, blood pressure, and so forth, is a particularly complex problem. This is so because there is no strict relationship between the two. Nevertheless, converted, for example, oral readings provide clinicians with access to their experience, an invaluable tool in dealing with the patient's disease.

Alternatively, measurement device 112 may collect a temperature reading every two seconds and store it onboard the battery-powered temperature sensor 112, and transmit the stored temperature reading every five minutes, for example to the patient's smart phone. This temperature measurement is repeatedly transmitted every two seconds by the Bluetooth transmitter onboard device 112 until a new one is read at the five minute mark while transmitting fuller data once each hour. For example, fuller information (for example more parameters or other information) respecting the prior readings communicated by over the Internet may be sent. As another example, if the patient had moved to a position where there was no connection to the Internet, or even the smart phone, information would be missing from the transmissions made over the Internet every five minutes during the previous hour. Indeed, there may be missing five-minute transmissions. During the once per hour transmissions, all data stored on board the inventive temperature sensing device 112 may be downloaded to the server over the Internet. Alternatively, only data which was not previously transmitted may be downloaded to the server over the Internet. In one embodiment the mobile application or the gateway application keeps track of the missed data. In order to save power the rules for scavenging data from the temperature sensing device is part of the device that collects data in the system.

A suitable conversion equation can be generated using clinical data.

Readings may be collected from skin sensor devices, oral temperatures taken one for each of the readings collected from skin sensor devices, oral temperatures plotted against their corresponding skin sensor reading, and a least-squares fit used to generate a linear relationship from the statistical data, or to vent to a sigmoid or other higher-order function.

Alternatively, any conversion equation may be transformed into a lookup table stored in memory (or a selected data pair set used directly), and the equation applied by looking up the input variable or variables stored in the lookup table and outputting the associated equation output parameter.

In accordance with the invention, a first temperature transducer facing the skin may be associated with each temperature measurement device 112. In an alternative preferred embodiment of the invention, a second transducer may be mounted on temperature measurement device 112 with its sensing surface facing outwardly to detect ambient temperature. The second transducer should be thermally isolated from the skin and the first transducer. The two temperature readings, namely the skin temperature reading from the inwardly facing first transducer and an ambient temperature reading from an outwardly facing second transducer may be combined to calculate heat flux losses between the skin and the ambient, and using this information to improve the measurement of core temperature. Ambient temperature may also be used to add or subtract an adjustment factor to the oral temperature generated by the system. Likewise, ambient temperature may be used to add or subtract an adjustment to the temperature reading produced by device 112. In a somewhat different vein, ambient temperature, when it is too high or too low may be used to disqualify readings from device 112 and not use them for clinical purposes. Ambient temperature may also be used to add or subtract an adjustment factor to the oral temperature generated by the system. Likewise, ambient temperature may be used to add or subtract an adjustment to the temperature reading produced by device 112. In a somewhat different vein, ambient temperature, when it is too high or too low may be used to disqualify readings from device 112 and not use them for clinical purposes.

In accordance with a preferred embodiment of the invention, temperature measurement device 112 comprises a circuit board with a CPU (including programmable firmware executing the method of the invention as illustrated in FIG. 4), a Bluetooth LE transceiver, flash memory, a battery, a 32 MHz clock (which clocks the functions of executing the instructions from the onboard flash memory and data collection from the sensor and the storing of information into the flash memory) and a 32 kHz clock (which may be used to preserve the system time of day), together with a serial multimaster communications bus coupling the listed operative parts of temperature measurement device 112 together. In accordance with the invention the time of day clock having a sleep mode is employed. In addition, the temperature sensor, in accordance with the invention, is only active when a reading is being taken, otherwise it is in sleep mode.

In accordance with the invention, the time of day clock is used to establish the taking of a measurement, for example, every five minutes. Optionally, the system may provide for taking measurements either between shorter or longer intervals. Such will allow for different degrees of power saving. The time of day clock is also used to reject unwanted requests from unauthorized users, and/or authorized users that have exceeded their allowed connect time, which may optionally be set at 20 seconds per day. This can be controlled at the server for example through a mobile application or gateway application.

As discussed above, in accordance with the invention, every five minutes, the clock on measurement device 112 at step 192 (FIG. 4) triggers the system at step 194 to wake up the microcontroller in temperature measurement device 112 (which was previously “sleeping”) at step 192. The system then proceeds to step 196, where the sensor is turned on to get it ready to produce a temperature reading. The system waits a very short period of time at step 198 for the turned on sensor to stabilize (typically on the order of 36 milliseconds), enabling the taking of an accurate temperature reading. At step 200, the microcontroller reads the sensed temperature, saving it to onboard flash memory on measurement device 112 at step 202.

The system then proceeds at step 204 to set up the Bluetooth advertising beacon with the temperature readings, the device unique ID and the Wi-Fi MAC address.

Next, after at step 204, the onboard CPU in temperature measurement device 112 is returned to sleep mode at step 206 after the addition of another temperature data point to onboard flash memory at step 202, in preparation for the transmission of a plurality of temperature data points at step 208. After step 204, the onboard CPU in temperature measurement device 112 resets the five minute timer and is returned to sleep mode at step 206. Similarly, the system is removed from sleep mode before downloading data over the Internet, and placed into steep mode again to conserve power after the data has been downloaded. The transmission of temperature data points at step 208 includes transmission of the time of the measurement as well as the unique identification number of the transducer on the temperature measurement device that here to the chest of the patient.

The return of measurement device 112 into sleep mode is a piece of the inventive strategy of conserving power. The above read and storage steps are repeated every five minutes onboard measurement device 112, for example as dictated by the clock at step 192. After the above periodic five minute reading/storage/process has been repeated for one hour, the system proceeds to step 210 which triggers the waking up of the controller at step 211, and the transmission at step 208 of the data collected over the previous hour.

As alluded to above, and tediously software is located and processing done on parts of the system other than temperature measurement device 112 which is battery-powered and adhered to the chest of the patient. The result of such architecture is the conservation of power provided by the onboard battery in device 112. For example, numerous functions may be located for example at the central server. The central server can do the processing associated with acceptance, rejection and modification of temperature measurements on account of any desired factors. Likewise, conversion of temperature measurements to oral temperature can also be done at the central server.

In accordance with the invention, when the Bluetooth transceiver on device 112 is not active, the Bluetooth transceiver is sleeping and consuming minimal power.

The functions of temperature measurement device 112, in the interest of limiting power consumption and maximizing battery life, are limited to the above functions of transceiver and temperature measurement sleeping and waking up; as well as sensor turn on, stabilization period execution, measurement saving, clock keeping and Bluetooth transmission. Generally, it is noted that interrogation of temperature measurement device 112 is not allowed or at least minimized in accordance with the preferred embodiment. In accordance with the preferred embodiment for other functions are performed at the server but without compromising the longevity of temperature measurement device 112, some functions may be moved to the clinician's computer patients smartphone.

In accordance with a preferred embodiment, means may be provided for system server 126 to interrogate device 112. However, in the interest of conserving power, the number of interrogations may be limited, for example to six per day or once every four hours and limited to only missed data. The mobile application keeps track of missed data and if the sensor and mobile application missed 12 of the 48 readings in a 4 hour period the mobile application or gateway application would only ask for the 12 missed readings, for example.

The present invention is of particular value with respect to the detection of sepsis. Sepsis is a condition which occurs when the body releases biochemicals into the bloodstream in response to and in an effort to fight infection. When these chemical agents are released, they can trigger inflammation throughout the body. The result is a cascade of changes that damage multiple organ systems, ultimately leading to organ failure. If sepsis continues unchecked, sometimes death will result, and this can happen in as little as 12 hours.

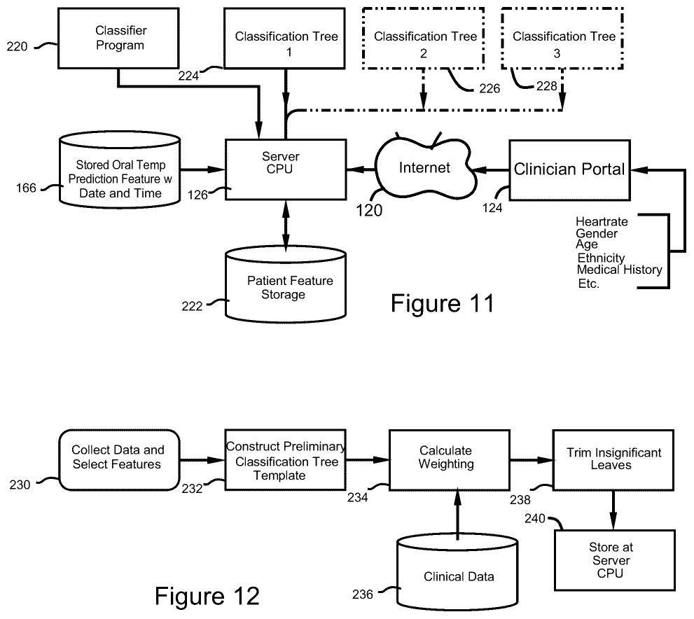

The temperature output of the apparatus of the present invention, as illustrated in FIGS. 1-3, may have its temperature output utilized in an artificial intelligence computer program. Such an inventive apparatus is illustrated in FIGS. 11 and 12, and may be used to provide reliable predictions of the future onset of sepsis in the patient.

Referring to FIGS. 11 and 12, use of a “classification tree” may be implemented to detect the likelihood of an imminent and present danger of the onset of sepsis. The inventive process assigns features to each node on a classification tree. In addition, the process comprises assigning a measure of the degree of causality of the feature at the node (“weight”). In addition, the process of the invention comprises allowing data to “flow” through the tree with a resulting output to be compared to known (clinical) data, preferably a very large collection of data as can be obtained from, for example, the National Institute of Health in the United Kingdom (“NIH”). This allows waiting to be assigned at each node.

Moreover, the accuracy of the process may, optionally, be continuously improved by adjusting the weighting factor at each node with additional data which may be obtained operating the inventive system, or maybe obtained from other sources.

The classification tree generated in this manner thus comprises the heart of an algorithm which utilizes the above mapping of skin temperature to oral temperature and may be used to predict the onset of Sepsis based not just upon temperature but based upon a broad set of “features” of the patient such as heartrate, gender, age, ethnicity, medical history, medications (available as EMR data) being taken etc., as well as the oral temperature noted above.

The invention is believed to provide a superior prediction algorithm because clinical data is largely based on oral temperature readings. Thus the generation of a meaningful oral temperature as described above in accordance with the invention has several advantages. As noted above, it correlates to clinical experience. However, and importantly so, the oral temperature reading generated by processing of the skin temperature reading collected in accordance with the present invention is superior to an oral temperature reading, because such readings suffer from numerous inaccuracies to to individual circumstances, as discussed above. Thus while the taking of oral temperatures builds a reliable knowledge said for the clinician, that knowledge said is a product of an overall assessment by the clinician of many different reading some of which may be more accurate than others, but all of which together provide and ask an informative professional experience data set. When that data set is assessed by the clinician, perhaps on an intuitive basis, the more meaningful oral temperature generated by the inventive system becomes more valuable than an oral reading in an individual case.

This advantage of the predicted oral temperature of the present invention, for example in accordance with the equations noted above, is thus to provide a more reliable input to the clinician.

Just as importantly, the more reliable oral temperature prediction of the present invention, when used as an input to an artificial intelligence system in the evaluation of the condition of the patient and the likelihood of the development of sepsis has the same advantages, and so far as the artificial intelligence system is based on the gross collection of temperature and other data features. More particularly, the temperature features collected in a large data set such as that of the NIH are based on oral temperatures taken by clinicians.

The inventive oral temperature protection is the product of a large data set, and as such, is more reliable when being used in artificial intelligence system to assess a patient's condition and the likelihood of the onset of sepsis using an artificial intelligence algorithm trained on a large data set.

Returning to the structure of the present invention, thus, the algorithm classification tree has nodes with each feature assigned to the node with each node being assigned a “weighting” as to the degree of “influence” that feature has on the set of patients developing sepsis. Given one can identify a large database with historical data about subjects developing Sepsis and information about what features each such patient had, the classification tree is thus, in accordance with the invention, “trained” to develop the correct weighting factors and then use with an input comprising, for example, the inventive physiological oral temperature. In addition, the classification tree may be “pruned” and different trees “combined” or “averaged” together. One such method is referred to as boosting and may involve such things as averaging many trees, where each is grown to related versions of the training data. For example, weighting decorrelates the trees, and may cause focusing on regions missed by past trees. In effect, a final classifier is effectively implemented with an implementation of a weighted average of classifiers.

Returning to FIG. 11, in accordance with the invention, a computer, for example server CPU 126 is program with a classifier program 220, which contains instructions which cause server CPU 126 to receive various patient features, such as heart rate, gender, age, ethnicity, medical history, and so forth for the particular patient a evaluated for the likely onset of sepsis. Such information may be input by a clinician at clinician portal 124 and sent over the Internet 122 server 126. These features are stored in a patient feature database 222.

As server CPU 126 is provided with each temperature reading of, for example, oral temperature, by the system over Internet 20, server CPU pursuant to the instructions in classifier program 220 retrieves the stored patient features and database 222 and processes the stored patient features, other than oral temperature, together with the current reported patient temperature, inputting the same into classification tree 224, and processing them in accordance with the instructions of program 222 produce a score, for example, the percentage likelihood of the patient developing sepsis in, for example, six hours, a day or other time period.

Optionally, multiple classification trees, such as additional trees 226 and 228 may be used in the evaluation implemented by classifier program 220.

As alluded to above, classification trees, such as classification trees 224-228 may be generated using the methodology illustrated FIG. 12, starting with the collection of data at step 230, for example and advantageously the collection of oral temperature data using the apparatus of the present invention. In accordance with the invention, efforts of others in collecting such data, such as the NIH. In accordance with the invention, the data collected at steps 230 and access from existing databases at step 236 is then allowed to flow through a preliminary classification tree constructed at step 232. At step 234, regression is used to examine causality between each factor and sepsis, and the same assigned as a weight in the weighting of each node in the classification tree. Less significant nodes are trimmed at step 238 and stored at step 240 in databases associated with CPU 126.

In accordance with the invention, output temperature data is encrypted, for example on onboard temperature measurement device 112 or (with better power consumption efficiency) external to the temperature measurement device 112, for example in the smart phone 114 associated with the patient. Performance of encryption as well as other functions on smart phone 114 is in keeping with the objective of minimizing power consumption loads on device 112.

In accordance with the invention, smartphone 114 and server 126 collect data originating from the advertising beacon on the Bluetooth transceiver. The server optionally (or alternatively the patient smart phone or software on the gateway) also includes software which keeps track of missing data and makes decisions as to when to wake up the processor to retrieve more data, but minimizes the same because this step is a relatively large power drain, and missing data may not be of urgency. In addition to the preferred embodiment where server 126, clinician portal computer 124 and practitioner devices 116 and 118, as a group, can only request missing information four times a day, it is believed that additional limitations as to how much information can be retrieved at a time are advantageous, for example limiting a request to the past 24 hours of data, or even the last hour of data.

In accordance with the invention, server 126 can, for example, change the five minute period clock to allow more frequent or less frequent data collections by commanding the mobile application or the gateway application to set the parameter on the temperature sensor.

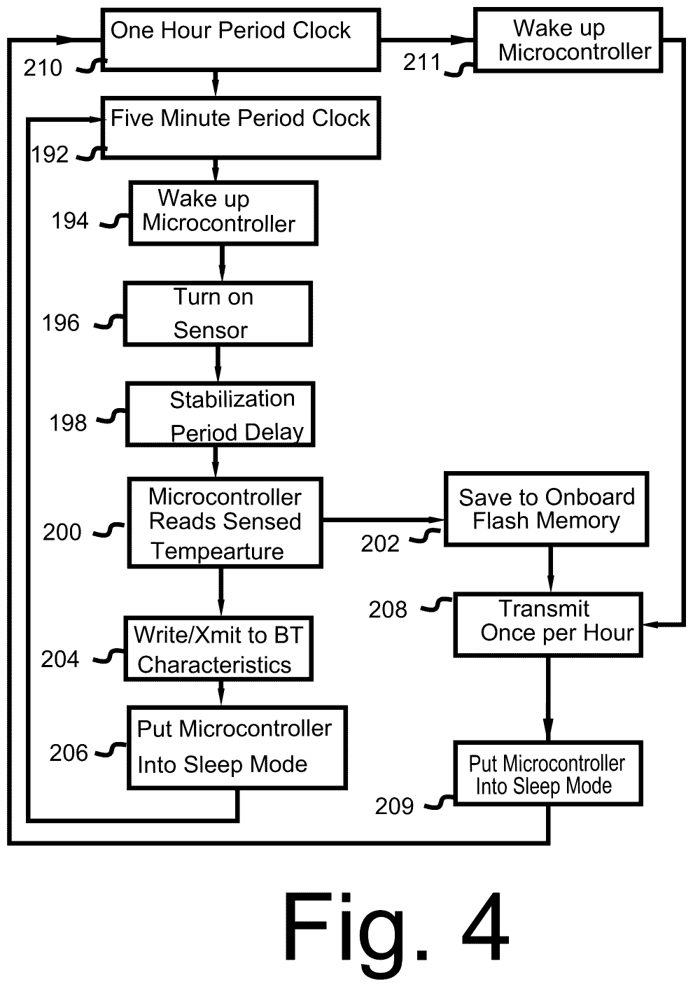

Referring to FIGS. 5-9, in accordance with a particularly advantageous implementation of the present invention, ambient temperature is measured. FIG. 5 is an exploded perspective view of a preferred embodiment of an inventive temperature measurement device 112 (which measures both skin temperature and ambient temperature), and which comprises a cutaneous information device (“CID”) 300 for use in the method and system of the present invention, for example, as described above. CID 300 comprises a sensor PCB (printed circuit board) assembly 302, which is connected to PCB pressure sensitive adhesive 304.

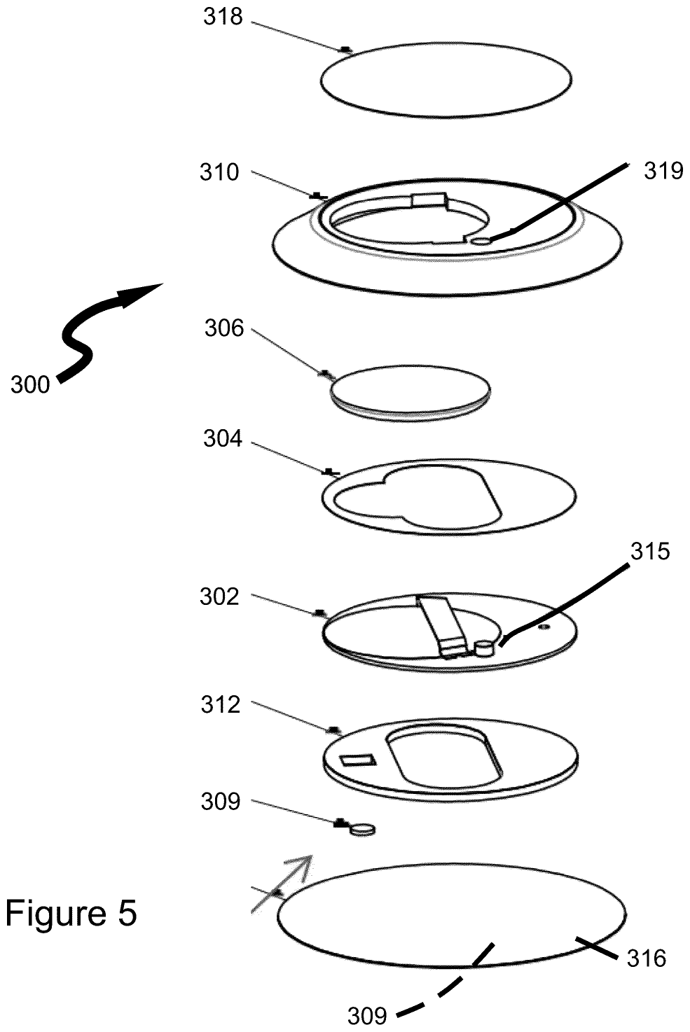

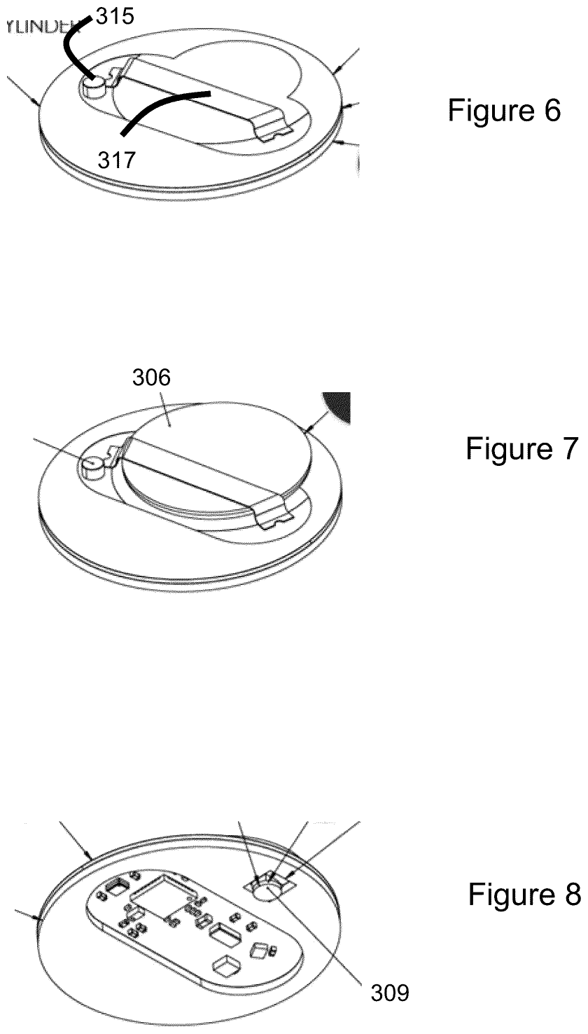

The operative parts are assembled and positioned in the frame 310 of the sensor module 300. Frame 310 is optionally made of a flexible polymeric material, such as a silicone plastic or rubber. On the human skin side 308 of the PCB 302 is the skin side temperature (Tsk) sensor 307 which is coupled through a heat pipe 309, which is a cylinder of metal for conducting heat. The heat pipe is placed against the sensor (not illustrated in FIG. 6), and the air gap between the inner seal is filled with thermal paste. More specifically, Foam filler 312 is placed on the bottom of the circuit card to fill any air gaps, and then ambient temperature sensor heat pipe 314 is inserted into an opening in foam filler member 312, and any remaining air gaps (for example between the sensor and that heat pipe and between the heat pipe and the thin plastic skin contacting inner seal member 316) are filled with thermal paste (not shown in FIG. 5). The result is that ambient temperature sensor 313 is well coupled to ambient temperature sensor heat pipe 314, which in turn is well coupled to the ambient. A thin and substantially temperature conducting PET inner seal assembly 316 is applied with foam filler 312 and sealed to frame 310. Final assembly to ensure waterproofness includes a thin plastic PET cover 318 (optionally made of PET and 1 mil thick), optionally a cover with printed information on its outer surface (not shown). Battery 306 is connected to frame 310, connected to a bottom skin side seal assembly 316 (which may be as simple thin circular disk of film), connected to foam filler 312, connected to the PCB (printed circuit board) 302 with PSA (pressure sensitive adhesive) 304 and thermal paste.

The construction is waterproof to IP 57 per IEC 60529. This marking could contain individual-specific information which can be visually seen, read, or scanned for patient identification, interaction, information exchange, and instructions and a non-contact communication device. The construction is waterproof to IP 57 per IEC 60529.

FIG. 6 shows a top view of the assembly showing a thermal stud battery clip 317. FIG. 7 shows battery 306 in place. A bottom view of the assembly is provided by FIG. 8 which shows the thermal stud, thermal paste, microcontroller, 32 MHz clock, and a 32 KHz clock on the printed circuit board. The printed circuit board also supports two designed-in antennas of conventional design, namely an NFC chip and a Bluetooth antenna, also conventional design and including an impedance matching balun. To remove any air gaps, thermal paste is inserted just above and in between all junctions that interfaces of the thermal sensor, but heat pipe and the outer housing film member for both the skin side facing thermal sensor 309, and the outward facing ambient temperature sensor 315 and its associated hole 319 (FIG. 5).

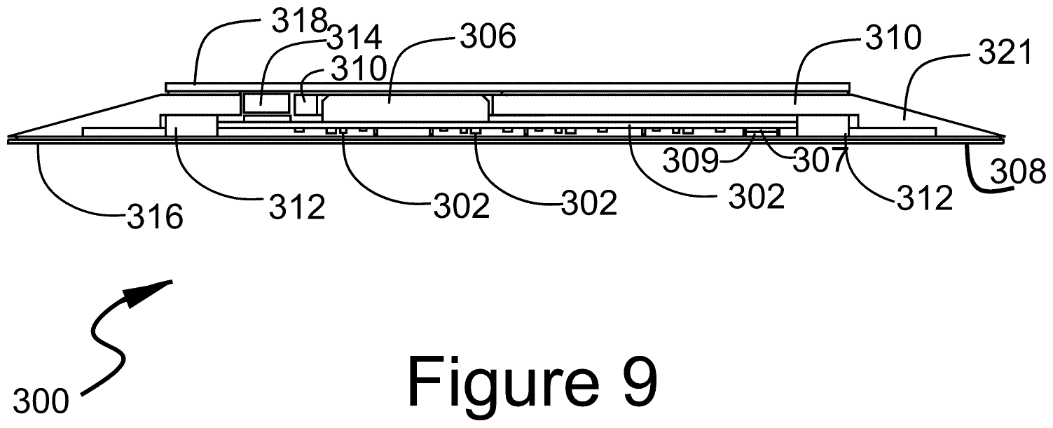

Referring to FIG. 9, a schematic representation of an inventive temperature measurement device is presented to more fully illustrate the embodiment of FIGS. 5-8. The same comprises a frame 310 which includes a thinned out peripheral portion 321 as illustrated most clearly in FIG. 9. This thinned down portion provides for a flexible and displaceable connection between the outer periphery of frame 310 and its mounting to the rigid PCB board 302. Thinned out peripheral portion 321 thus accommodated movements of the skin of the patient and results in reduced stress being applied to the adhesive holding temperature measurement device 112 to the skin of the patient.

In accordance with the preferred embodiment, it is important that both the inward skin facing transducer measuring skin temperature and the outward facing ambient temperature transducer measuring the ambient temperature be isolated from each other by being separated from each other and/or by being insulated from each other. Likewise, the ambient temperature measuring transducer should be generally isolated from the skin. Also, the skin temperature measuring transducer should be isolated from the ambient.

In preferred embodiments, both have RF matching circuits for proper operation and maximum efficiency. Under one thermal stud is the ambient temp sensor (not shown), and under the skin temperature sensor heat pipe is the skin temperature sensor (not shown).

In accordance with the invention, it is contemplated that different-sized CIDs may be used for larger and smaller people as well as varying application locations for the CID based on the environment. In a preferred embodiment, the device is less than about 6 grams, no greater than 10 gm, 40-50 mm in diameter, preferably less than 44 mm in diameter, and all materials comply with ISO-10993 biocompatibility.

The CID is constructed to be optimized for size, weight, power, and flexibility as a skin-applied device. The accuracy of temperature measurement is critical, and air gaps are typically problematic when trying to measure the precise skin temperature, and when present, make the production reliability (CID to CID variances) inconsistent. The components in the event of temperature sensor 112 must also be sealed to exclude moisture either from external sources or that which is naturally present on the skin, such as sweat. The inventive temperature sensor 112 uses on the skin side a thermal stud or pipe for coupling temperature to the temperature transducer and thermal paste to remove all air gaps in the temperature sensing path and thus provide a good thermal path to the skin-side layer in the case of the skin temperature sensor. Similarly, thermal paste is use in the thermal path to the ambient facing side of the inventive temperature sensor 112. To minimize the storage requirements and the processing requirements, only the measured temperature is transmitted to the DFA (i.e. the overall software being used by the system). The DFA will then perform the necessary calculations required to convert skin temperature to core-body temperature.

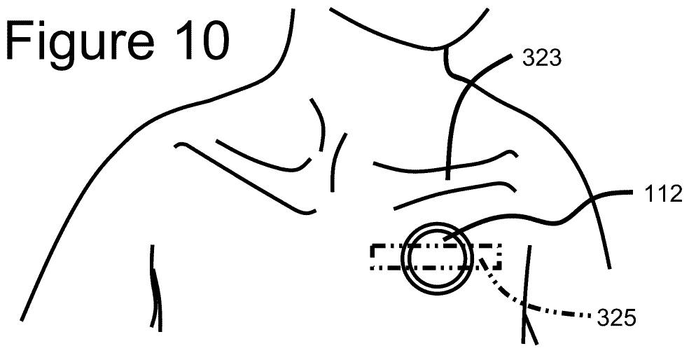

Referring to FIG. 10, the inventive measurement device 112 is illustrated adhered to the chest of the patient by a strip of medical grade adhesive bandage 325. In accordance with the invention, it is positioned below the left clavicle 323 of the patient. If desired multiple strips of bandage may be used. However, care should be taken not to place bandage strips over the outwardly facing ambient temperature measurement transducer, in order to promote the exposure of the transducer to the ambient. In accordance with a preferred embodiment, the location of the outwardly facing ambient temperature transducer may be marked visually and bear a warning not to cover with a bandage. When ambient temperatures are above 80° Fahrenheit or below 65° Fahrenheit, there is a high likelihood that an inaccurate reading is being received, and the detection of such ambient temperatures may be used to trigger the discarding of the associated skin temperature measurement.

In accordance with the invention, ambient temperature may be used as an input to the methodology of the invention. More particularly, in accordance with the invention, data is periodically sent from the patient over the system illustrated in FIGS. 1-3 to, for example, the central server for processing of that data in accordance with the criteria (for example criteria specified by the clinician), and physiological information, for example, body temperature, is sent to the clinician periodically and/or in response to detected emergency conditions, or in response to specified criteria.

Optionally, the computing logic as above for determining authorization, temperature conversion and data scavenging may be all centrally located in one logic module (“Data Fusion Aggregator” or “DFA”) and may be located remote from the central server. The logic module can be placed, for example, on a local PC with proper radio connectivity to the sensor or sensors on the human body. The logic module can be configured within a Bluetooth gateway to the central server. Alternatively, the logic module can be incorporated into a mobile application, or can be placed in a custom device and worn on-body for storage and retransmission to the central serverIn . . . .

In accordance with the invention, methods for initial authentication and subsequent reauthentication have been described above. Activation of the inventive temperature measurement device 112 (cutaneous information device or CID) and associated system is initiated by a person providing identifying information to be associated with the CID in the associated inventive system. The information can be communicated to a central database/processor directly or via a local server/processor through, for example, a plurality of Internet connected computers, or, as illustrated, cellular smart devices (such as smartphones). Cellular smart devices are connected, via cell towers and cyberspace to a central server. Upon the initiation of communication with a particular CID with the central server, information is checked to determine whether the CID is already registered on the system or whether a new patient record needs to be created. After the patient is determined to have been registered on the system, or a new patient record created, the CID is applied to the surface of the patient's skin, for example using an adhesive layer associated with the CID . . . .

Information collected by the CID of the patient is then passed to the DFA and may be further processed using data not available to the CID to enhance the accuracy and validity of the data. This is done as described in detail above, using such AI techniques as and not limited to neural networks, linear regression generative AI models, associated with the demographics in the patient record. The demographics may include physiological information, age weight, height, medication, disease state and other information. This data predictive or analytical is passed to the patient record for the physician to analyze and prescribe further treatment. Access to the information contained on the central server is desirably managed by security protocols to ensure that the information being provided is on a need-to-know basis. While cloud connection is advantageous, continuous cloud connection is not necessary for functionality. As an alternative to continuous connection, the device comprising the DFA, as an alternative to a continuous connection phone may be put into low power mode, where data is time stamped and stored locally on the device, and then uploaded in due course according to a DFA-CID pre-determined-protocol for communications. For use in a closed setting (e.g. hospital, rehab facility, cruise ship), local connection to a centralized server may be sufficient where the DFA-CID connection may be done via a gateway device, such as a Bluetooth-wifi gateway which communicates with the CID and the local centralized server can contain the DFA as described above, or the DFA can be incorporated into the gateway.

The data fusion aggregator, in accordance with the invention, is implemented as software, for example, on a smart phone with which the CID is in direct communication. The architecture of the DFA comprises multiple logical units. These units enable accurate data computation from the raw sensor data in combination with preprocessed data. For example: The data coming from the sensor is skin temperature. The DFA is enabled with one of several algorithms; machine learning, basic translation from skin to core, as well as AI techniques that would otherwise consume power in the CID. The DFA is to provide a mechanism for connecting for authorized sensors on the network and pass processed data to the cloud with the device signature. The DFA is configured to communicate with the sensor, do data management, as per the design of the sensor. This allows the sensor to save power in data processing, and management, and allows the sensor to send privately encrypted data without having to turn on the sensor receiver and wait for bi-directional communications to take place. The communication and processing optimization is done on a sensor-by-sensor basis, and the DFA communicates and processes data and is, optionally but advantageously, unique for each type of CID (sensor) in the system. Each CID type has a unique data protocol, and access needs. For example, the data transmission needs and data payloads for a temperature sensor are much less than EKG and arrhythmia detection. Thus, these are two different CIDs require two different DFAs for each CID to operate. There can be CIDs with multiple sensors such as temperature, heart rate, and accelerometry, in this instance the DFA will be able to manage each data stream as configured by the central server and perform the conversion of sensor information to useable data by a clinician or user application.

The DFA manages the security, non-physical system frangibility and communication channel between the sensor and the cloud and processes only sensors that have been authorized to be on the network. The DFA identifies a CID but requires authentication from an upstream device/server to enable communications between the DFA and the CID. The DFA is the device that is authenticated to receive specific CID data. The data from each CID is time stamped and each DFA can report to an application that can then time-correlate the data.

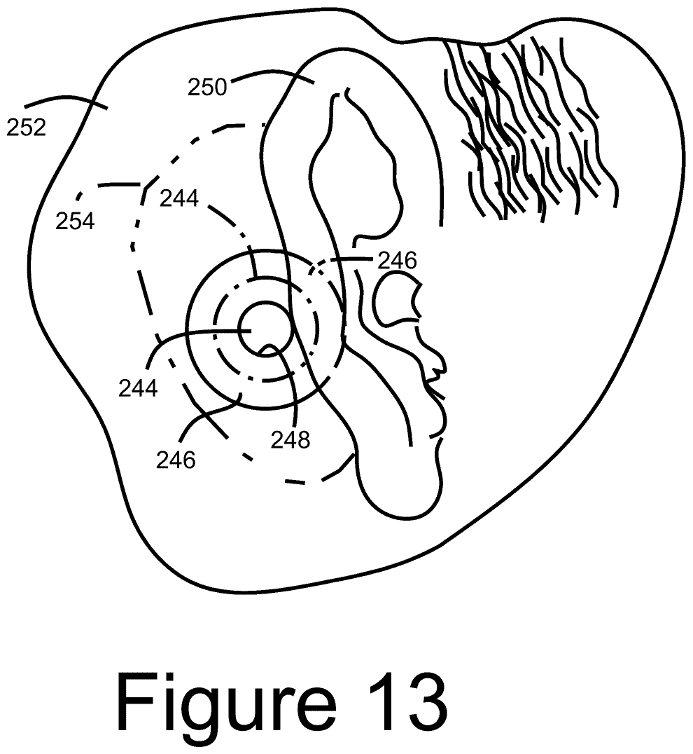

The DFA functionality can also accept settings from the central server that are applied to enhance the precision of the sensor functionality. Settings can be age, gender, frequency of measurements, weight, height to mention a few. Settings can include non-physiological conditions such as environmental conditions. This decision making happens above the DFA and CID, and is driven by the specific applications looking for specific results. For example, a sports or fitness application may have two sensors, one temperature CID and the other a heart-rate CID, or a CID coupled to the mastoid bone to detect shock during a game. In accordance with the invention, the transducer for detecting shock to the skull by coupling to the mastoid bone is positioned behind the ear roughly midway between the top and bottom of the ear. Such a shock detecting sensor is illustrated in FIG. 13. It is anticipated that upon the detection of mechanical shock to the head (if a shock threshold is exceeded) cooling of the head may be very promptly implemented and the injury limited. For early detection, monitoring of temperature, in combination with monitoring mechanical shocks to the mastoid, is believed to be of importance, in so far as the human body is sensitive to shock. The response of the human body to shock is believed to have an effect on other physiological parameters, such as, the rate and other characteristics of heartbeat, and such response, consisting of a number of physiological parameters may be used as input features to an artificial intelligence algorithm in order to provide an enhanced detection of dangerous conditions, as opposed to detection of the seriousness of shock relying only on measurement of mechanical impacts to the skull. In accordance with this embodiment of the invention, for example, heart rate, temperature, who measured impact shocks may be used as features in an artificial intelligence algorithm trained on clinical data for some or all of these features and head trauma outcomes. When a serious impact is thus detected, the skull may be put in a cooling jacket to reduce the term effects of impacts to the skull. Such multiple sensors may send signals to a single transceiver, such as a smartphone, and individual signals may be processed by a single algorithm, or multiple algorithms may be used to monitor for different conditions.

The two DFAs communicate to an application upstream for the sensor system. In accordance with the invention, it is contemplated that the provision of a mastoid shock/concussion detector will be provided with the time information associated with the concussion or shock, and that this time information will be presented to the clinician or team doctor who can look at shock data and temperature data, for example, and make a clinical judgment knowing the time during which both measurements were made and thus judge how they are related to each other and, more importantly, how they may suggest remediative approaches. In accordance with a particularly preferred embodiment of the invention, a so-called nine-degrees of freedom transducer will be coupled to the mastoid bone. Mechanical transducers outputting multiple signals are available thus enabling cost-effective implementation of the technology of the invention. More particularly, such devices include detection of acceleration in the X, Y and Z directions, as well as three degrees of rotational freedom, in addition to position in X, Y, and Z space. More particularly in accordance with the preferred embodiment, it is contemplated that a mastoid mounted nine degree of freedom transducer will output a low-power RF signal and that this signal will be received by a CID mounted on, for example, the chest of the player and then be relayed to the system for display to a clinician or team doctor. It is anticipated that the CID relaying the nine parameters will transmit to, for example, a smart phone or computer by way of Bluetooth, and that the relay device will be maintained in close proximity, or at least close enough proximity to allow the transmission. In principle, antenna structure can be included in the playing field or multiple receiving devices positioned around the playing field to facilitate the relay of information from the mastoid sensor to the CID to the system and the clinician on hand at the game, or through other potential channels. In accordance with the preferred embodiment, the absolute position of the shock sensor 244 may be determined by integrating the output of the accelerometer to get velocity, and then performing another integration operation to derive absolute position on the playing field and orientation represented by position along three rotational axes. More particularly, referring to FIG. 13, a disk shaped shock sensor 244 is held in place by a circular bandage 246 with a hole 248 in the middle of it in order to allow shock sensor 244 to shed heat. In accordance with the invention, it is contemplated that mastoid sensor is coupled into the inventive system as illustrated, for example, in FIGS. 1-3, above. The assembly of shock sensor 244 and bandage 246 is positioned under the middle portion of the rear of the ear 250 of the user, as illustrated. The result is that shock sensor 244 is directly above the hard surface of the skin 252 of the user. That hard surface is created because the mastoid bone 254 is positioned underneath that portion of the skin 252 of the user where sensor 244 is placed. The temperature DFA-CID captures core body temperature from normal living. However, as literature has indicated (U.S. Pat. No. 10,702,165-B2 to Buller), when heart-rate reaches a certain level above everyday living for extended periods of time with appropriate algorithms the heart-rate is a better indicator of elevated temperatures of the core vs. skin-to core temperature derivation. At this point the upstream application can make the decision to use the heart-rate to core conversion until-such a time that the heart rate returns to normal. The heart-rate to core temperature conversion relies on knowing what the pre-activity core body temperature was, and the temperature CID was providing that information.

Therefore, a simple heart-rate monitor, and a skin-to-temperature monitor together become a high-performance sensor without having to sacrifice power, calculation to achieve the superior performance.

In preferred embodiments, the DFA module comprises a secure software package that has a simple API (application programming interface) such that it is easily integrated into smart devices with the appropriate communication paths, and processing power. The DFA can reside in smart-watches with internet connectivity, mobile handsets, WIFI and cellular gateways, personal computers, real-time processors and can be implemented in the cloud.

Sensor placement on the body or in home varies depending on the functionality required. Thus, in some instances one may have multiple sensors on the body and want to combine sensors to enable a higher level of diagnosis. Some sensors are not on the body and that data may want to be combined with a patient, such as room temperature from a smart thermostat, the weight scale in the bathroom, blood pressure monitor, pulse oximetry, blood-glucose, sleep sensors in a mattress etc. All these sensors can be authorized as belonging to a single or multiple individuals through a higher-level authorization module that can be communicated to the DFA. For example, a central server (which is capable in accordance with the preferred embodiment of servicing a number of individuals) may be configured to have five devices assigned to an individual. Each Device has a DFA unit connected to a CID. These are configured to be securely identified to a single user. When the user connects the devices to his/her person the server application authenticates the CID sensor and DFA as belonging to the person. This association can be done via a near-field communication radio (4-inch transmission distance) and the device is securely connected to the server. Thus all the sensors in the system may have NFC identifiers and one system with multiple DFA and CIDs can be registered to one unique individual. The application that sits above the DFA and CIDs, and coordinate the data, as in the to make a more valuable data set. This can be done in multiple situations. With an activity sensor collecting stride information and combining it with the inter-beat-interval will provide a fitness index. The design of two CIDs is simple and, in both instances, requires very little power. Combining a heart-rate sensor and a stride sensor and time-correlating information for both will cause the stride sensor to consume more power as the ekg sensor or SpO2 sensor will consume more power than the stride sensor and the combined sensor will consume more power thus requiring larger size and weight. In the case where two temperature CIDs are used the DFA at the time of configuration on the body location will be identified to the DFA, and the DFA will be configured to utilize both sets of data processing with algorithms, machine learning, or other AI and data analytical techniques.

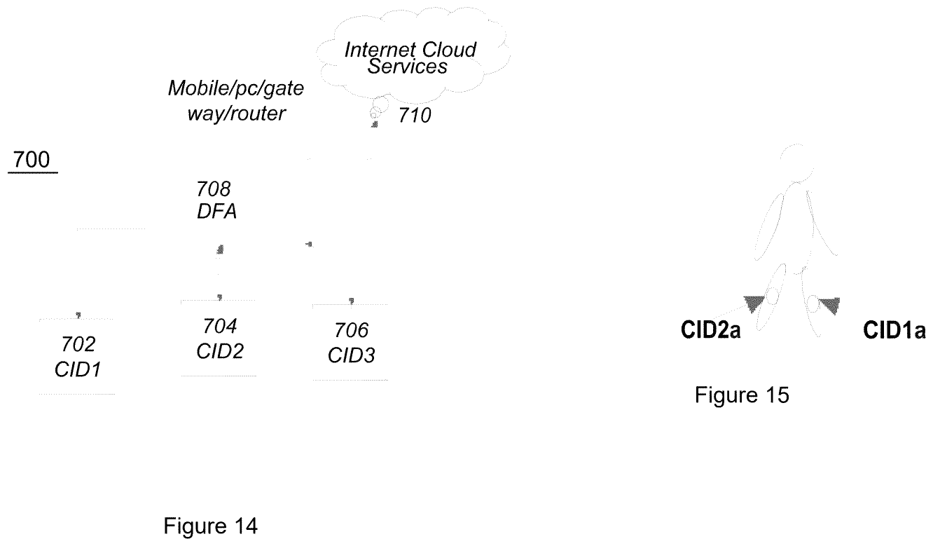

Referring to FIG. 14, system 700 is an overview of the system comprising multiple CIDs 702, 704, 706 that are connected each to a data fusion aggregator (DFA) 708 which can be housed in a smart watch, laptop, mobile device, gateway set top box. DFA 708 would have the capability to connect to multiple CIDS, as well as a server connection point 710, which could connect to cloud services which could be remote or within a localized network (e.g. hospital, cruise ship). One or more CIDs with dedicated DFAs can be used to measure various biometric data including temperature, heart rate, blood pressure, and other data points such as respiration, body positioning etc.

For example, referring to FIG. 15, a deep vein thrombosis (DVT System) patient may wear two CIDs to measure the temperature difference between the locations. Both CID2a and CID1a are connected to the same DFA. The DFA collects the time correlated data and can indicate when the temperatures are drifting apart and when one of the two CIDs exceeds a predetermined threshold determined by the clinician to alert the clinician and patient of a possible issue. The DFAs may receive skin temperatures data but not convert the temperatures to core-but pass the skin temperature, the skin temperature differences to the system. The system may then discern if the two sensors have the significant temperature change differential to indicate an issue with the patient.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed:

1. Apparatus for measuring temperature on a mammal such as a human, comprising:
(a) a temperature measurement device, comprising:
(i) an onboard device power source powering components of said temperature measurement device;
(ii) a clock having a first high speed output, and a second low speed output;
(iii) a data memory sector;
(iv) a digital processor responsive to said high-speed output to generate a storage trigger signal;
(v) a temperature measurement transducer responsive, to said storage trigger signal from said digital processor, to periodically collect a temperature measurement and to couple said collected temperature measurement to said data memory sector to store said collected temperature measurement in said data memory sector to accumulate data in said data memory sector as an accumulation of data in the form of a plurality of data points;
(vi) a wireless transceiver having an input and an output, said digital processor responsive to said low speed output to generate a transmission trigger signal and couple said transmission trigger signal to said data memory sector, said transmission trigger signal causing said data memory sector, to couple said accumulation of data stored in said data memory sector to the input of said wireless transceiver for transmission; and
(vii) a non-volatile memory sector with a program of onboard instructions for controlling said digital processor to cause said temperature measurement transducer to store said data points in said data memory sector to cause said wireless transceiver to transmit said data points; and
(viii) a chassis member supporting said power source, said clock, said data memory sector, said digital processor, said temperature measurement transducer and said wireless transceiver;
(b) a publicly accessible network;
(c) a wireless repeater receiving the accumulation of data output from said wireless transceiver and coupling the same to said publicly accessible network;
(d) a server coupled to said publicly accessible network to receive said accumulation of data, said server comprising:
(i) a server central processing unit;
(ii) a temperature data memory coupled to receive and store said data points; and
(iii) a non-volatile server program memory with a program of server instructions causing said server central processing unit to receive, store, and transmit to at least one user said data points; and
(e) a non-volatile temperature conversion program memory with a program of temperature conversion instructions for converting said data points to predicted physiological temperature readings for said transmission to said at least one user.

2. Apparatus as in claim 1, wherein said non-volatile temperature conversion program memory is not located on said chassis or powered by said onboard device power source.

3. Apparatus as in claim 2, wherein said predicted physiological temperature is a predicted oral temperature, and wherein said transceiver is a mobile personal computing device coupled to said publically accessible network, and the non-volatile temperature conversion program memory is coupled to and runs on said personal computing device.

4. Apparatus as in claim 2, wherein said wireless repeater is a smartphone and the non-volatile temperature conversion program memory is coupled to and runs on a digital signal processor on said smartphone.

5. Apparatus as in claim 2, wherein said predicted physiological temperature is a predicted oral temperature, and wherein said chassis member is a waterproof housing.

6. Apparatus as in claim 2, wherein said predicted physiological temperature is a predicted oral temperature, and wherein said program of temperature conversion instructions for converting said data points to predicted oral temperature readings was generated by collecting a plurality of collected clinical calibration skin temperature measurements from skin mounted temperature measurement transducers, and collecting a plurality of collected clinical calibration oral temperature measurements, said calibration skin temperature measurements and said clinical calibration oral temperature measurements comprising a plurality of data points, and fitting a function to said plurality of data points.

7. Apparatus as in claim 1, wherein said temperature conversion instructions for converting said data points to predicted physiological temperature readings was generated by collecting a plurality of collected clinical calibration skin temperature measurements from skin mounted temperature measurement transducers, and collecting a plurality of collected clinical calibration physiological temperature measurements, said calibration skin temperature measurements and said clinical calibration physiological temperature measurements comprising a plurality of data points, and fitting a function to said plurality of data points.

8. Apparatus as in claim 1, wherein said physiological measurement is core temperature, and wherein said temperature conversion instructions are a function of clinical skin temperature measurements and clinical core temperature measurements and further comprise oral temperature conversion instructions for converting core temperature measurement readings from said temperature measurement transducer to oral temperature measurement readings, said oral temperature conversion instructions based upon clinical skin temperature measurements and clinical oral temperature measurements.

9. Apparatus as in claim 8, further comprising a high temperature alarm circuit for signaling a fever condition, said high temperature alarm circuit being triggered by core temperature measurements exceeding a fever indicating threshold.

10. Apparatus as in claim 8, further comprising a high temperature alarm circuit for signaling a fever condition, said high temperature alarm circuit being triggered by core temperature measurements or converted oral temperature measurements exceeding a fever indicating threshold at least twice in sequential readings.

11. Apparatus as in claim 1, wherein said onboard device power source comprises a power source selected from the group consisting of a battery or a capacitor.

12. A diagnostic tool as in claim 1 for generating an early prediction of the likely onset of sepsis in a patient, comprising a classifier, and a set of diagnostic features other than temperature associated with said patient, and oral temperature as a feature, and further comprising:
(f) storing in a non-transitory computer readable medium that stores instructions that when executed by the server central processing unit causes the server central processing unit to:
(i) provide the set of diagnostic features, other than physiological temperature, associated with said patient as an input to the classifier and also provide said physiological temperature of said patient as an input to the classifier, said physiological temperature being a feature, wherein the classifier is trained with data from a plurality of individuals who have had sepsis;
(ii) evaluate, with the classifier, the likelihood of sepsis by using, as an input to the classifier, both (I) the set of diagnostic features other than physiological temperature and (II) said physiological temperature feature; and
(iii) generate an output indicating whether there is an indication that the subject is likely to develop sepsis.

13. Apparatus as in claim 12, wherein said diagnostic features other than oral temperature comprise one or more features selected from the group consisting of heart rate, gender, age, and ethnicity.

14. Apparatus as in claim 13, wherein said physiological temperature feature is a predicted physiological oral temperature.

15. Apparatus as in claim 1, wherein the temperature measurement transducer faces in one direction in the general direction of the skin of the user and is coupled to collect a skin temperature reading, and further comprising an additional temperature transducer facing generally away from the skin of the user toward the ambient and is positioned to collect an ambient temperature reading, and wherein when said ambient temperature reading is below a low threshold value or above a high threshold value, said collected skin temperature reading is discarded or devalued.

16. Apparatus for measuring temperature, comprising:
(a) a temperature measurement device, comprising:
(i) a device power source powering components of said temperature measurement device; dependent battery or capacitor
(ii) a clock having a first high speed output, and a second low speed output;
(iii) a data memory sector;
(iv) a digital processor responsive to said high-speed output to generate a storage trigger signal;
(v) a temperature measurement transducer responsive, to said storage trigger signal from said digital processor, to periodically collect a temperature measurement and to couple said collected temperature measurement to said data memory sector to store said collected temperature measurement in said data memory sector to accumulate data in said data memory sector as an accumulation of data in the form of a plurality of data points;
(vi) a wireless transceiver having an input and an output, said digital processor responsive to said low speed output to generate a transmission trigger signal and couple said transmission trigger signal to said data memory sector, said transmission trigger signal causing said data memory sector, to couple said accumulation of data stored in said data memory sector to the input of said wireless transceiver for transmission; and
(vii) a non-volatile memory sector with a program of onboard instructions for controlling said digital processor to cause said temperature measurement transducer to store said data points in said data memory sector to cause said wireless transceiver to transmit said data points; and
(viii) a chassis member supporting said power source, said clock, said data memory sector, said digital processor, said temperature measurement transducer and said wireless transceiver;
(b) a publicly accessible network;
(c) a wireless repeater receiving the accumulation of data output from said wireless transceiver and coupling the same to said publicly accessible network;
(d) a server coupled to said publicly accessible network to receive said accumulation of data, said server comprising:
(i) a server central processing unit;
(ii) a temperature data memory coupled to receive and store said data points; and
(iii) a non-volatile server program memory with a program of server instructions causing said server central processing unit to receive, store, and transmit to at least one user said data points; and
(e) a non-volatile temperature conversion program memory with a program of temperature conversion instructions for converting said data points to predicted physiological temperature readings for said transmission to said at least one user, wherein said program of temperature conversion instructions for converting said data points to predicted physiological temperature readings was generated by collecting a plurality of collected clinical calibration skin temperature measurements from skin mounted temperature measurement transducers, and collecting a plurality of collected clinical calibration physiological temperature measurements, said calibration skin temperature measurements and said clinical calibration physiological temperature measurements comprising a plurality of data points, and fitting a function to said plurality of data points.

17. Apparatus as in claim 16 wherein said predicted physiological temperature is a predicted oral temperature.

18. Apparatus is in claim 16, wherein said program of temperature conversion instructions for converting said data points to predicted oral temperature readings was generated by collecting a plurality of collected clinical calibration skin temperature measurements from skin mounted temperature measurement transducers, and collecting a plurality of collected clinical calibration oral temperature measurements, said calibration skin temperature measurements and said clinical calibration oral temperature measurements comprising a plurality of data points, and fitting a function to said plurality of data points.

19. A diagnostic tool for generating an early prediction of the likely onset of sepsis in a patient, with a classifier, coupled to receive and operate on a set of diagnostic features other than temperature associated with said patient, and a derived temperature reading comprising a predicted oral temperature derived from a patient's physiological temperature as an additional feature, comprising:
(a) a central processing unit;
(b) a temperature measurement device producing a physiological temperature output; and
(c) a non-transitory computer readable medium that stores instructions that when executed by the central processing unit causes the central processing unit to:
(i) convert the physiological temperature output of said temperature measurement device to a predicted oral temperature reading, said set of instructions implementing a function between physiological temperature output and predicted oral temperature readings to generate said predicted oral temperature reading, wherein said function was derived by (A) collecting data comprising a plurality of sets of clinical oral temperature readings and associated clinical temperature readings more reliable than clinical oral temperature readings, and (B) fitting said data to derive said function;
(ii) provide the set of diagnostic features other than temperature associated with said patient, and the predicted oral temperature, associated with said patient as an input to the classifier, wherein the classifier is trained with data corresponding to data in said set from a plurality of individuals who have had sepsis;
(iii) evaluate, with the classifier, the likelihood of sepsis by using, as an input to the classifier, both (A) said a set of diagnostic features other than temperature associated with said patient and (B) said derived oral temperature as features; and
(iv) generate an output indicating whether there is an indication that the patient is likely to develop sepsis.

20. A method for measuring and monitoring temperature, comprising:
(a) taking a temperature measurement using a temperature measurement device, comprising:
(i) using a device power source to power components of said temperature measurement device;
(ii) operating a clock to produce a first high speed output and a second low speed output, said high speed output of said clock being at least 10 times as fast as said low speed clock output;
(iii) reading and writing to a data memory sector;
(iv) generating a storage trigger signal using a digital processor responsive to said high-speed output of said clock;
(v) periodically collecting a temperature measurement using a temperature measurement transducer responsive to said storage trigger signal from said digital processor, and
coupling said collected temperature measurement to said data memory sector to store said collected temperature measurement in said data memory sector to accumulate data in said data memory sector as an accumulation of data in the form of a plurality of data points;
(vi) generating, in response to said low speed output, a transmission trigger signal using said digital processor, and coupling said transmission trigger signal to said data memory sector, said transmission trigger signal causing said data memory sector to couple, for transmission, said accumulation of data stored in said data memory sector to the input of a wireless transceiver having an input and an output; and
(vii) using a non-volatile memory sector with a program of onboard instructions for controlling said digital processor to cause said temperature measurement transducer to store said data points in said data memory sector to cause a wireless transceiver to transmit said data points; and
(viii) using a chassis member to support said power source, said clock, said data memory sector, said digital processor, said temperature measurement transducer and said wireless transceiver;
(b) using a wireless repeater to couple, to a publicly accessible network, the accumulation of data output from said wireless transceiver;
(c) receiving said accumulation of data using a server coupled to said publicly accessible network, by using a server central processing unit to receive and store said data points using a temperature data memory coupled to said central processing unit of said server; and receiving, storing, and transmitting to at least one user said data points using a non-volatile server program memory with a program of server instructions; and
(d) predicting an oral temperature reading for said transmission to said at least one user using a non-volatile temperature conversion program memory with a program of temperature conversion instructions for converting said data points, wherein said non-volatile temperature conversion program memory is not located on said chassis or powered by said device power source.

21. A method for evaluating the likelihood of an individual developing sepsis or other disease or condition using a classifier, a set of diagnostic features other than temperature and a physiological temperature as features, said physiological temperature being a predicted temperature based on a function defining the relationship between oral temperatures taken by clinicians and a skin temperature-based electronically collected reading with the function being used to convert real-time physiological skin temperature measurements to predicted oral temperatures, comprising:
(e) storing, in a non-transitory computer readable medium, instructions that when executed by a central processing unit causes the central processing unit to:
(i) provide the set of diagnostic features, other than physiological temperature, associated with said patient as an input to a classifier and also provide said predicted oral temperature of said patient as an input to the classifier, said predicted oral temperature being a feature, wherein the classifier is trained with data from a plurality of individuals who have had sepsis;
(ii) evaluate, with the classifier, the likelihood of sepsis by using, as an input to the classifier, both (I) the set of 26. A method for evaluating the likelihood of an individual developing sepsis using a classifier, a set of diagnostic features other than temperature and a physiological temperature as features, said physiological temperature being a predicted temperature based on a function defining the relationship between oral temperatures taken by clinicians and a skin temperature-based electronically collected reading with the function being used to convert real-time physiological skin temperature measurements to predicted oral temperatures, comprising:
(e) storing, in a non-transitory computer readable medium, instructions that when executed by a central processing unit causes the central processing unit to:
(i) provide the set of diagnostic features, other than physiological temperature, associated with said patient as an input to a classifier and also provide said predicted oral temperature of said patient as an input to the classifier, said predicted oral temperature being a feature, wherein the classifier is trained with data from a plurality of individuals who have had sepsis;
(ii) evaluate, with the classifier, the likelihood of sepsis by using, as an input to the classifier, both (I) the set of diagnostic features other than physiological temperature and (II) said predicted oral temperature feature; and
(iii) generate an output indicating whether there is an indication that the subject is likely to develop sepsis.
diagnostic features other than physiological temperature and (II) said predicted oral temperature feature; and
(iii) generate an output indicating whether there is an indication that the subject is likely to develop sepsis.
